(19) United States
(12) Patent Application Publication
(10) Pub. No.: US 2026/0256412 A1
(43) Pub. Date: Sep. 3, 2026

(54) DEMENTIA-RELATED NEURODEGENERATION TRACKING USING MAGNETIC RESONANCE IMAGING (MRI)

(71) Applicants: Jennifer Bramen, Santa Monica, CA (US); Prabha Siddarth, Encino, CA (US); Gavin Thomas Kress, Knoxville, TN (US); Emily Shannon Popa, Discovery Bay, CA (US)

(72) Inventors: Jennifer Bramen, Santa Monica, CA (US); Prabha Siddarth, Encino, CA (US); Gavin Thomas Kress, Knoxville, TN (US); Emily Shannon Popa, Discovery Bay, CA (US)

(73) Assignee: Pacific Neuroscience Institute Foundation, Santa Monica, CA (US)

(21) Appl. No.: 18/793,712

(22) PCT Filed: Feb. 1, 2023
(86) PCT No.: PCT/US2023/012153

§ 371 Date: Aug. 2, 2024

Related U.S. Application Data
(60) Provisional application No. 63/306,760, filed on Feb. 4, 2022.
(60) Provisional application No. 63/407,575, filed on Sep. 16, 2022.

(51) Int. Cl. A61B 5/00; A61B 5/055; G06T 7/00

(52) U.S. Cl. CPC A61B 5/4088; A61B 5/055; A61B 5/7264; G06T 7/0012; G06T 2207/10088; G06T 2207/20081; G06T 2207/30016

(57) ABSTRACT

Systems and methods for tracking neurodegeneration biomarkers and assessing cognitive state. Implementations can include at least one computing device receiving magnetic resonance images, determining at least one regions of interest, and determining a benchmark based on a biomarker metric for a neurodegenerative disease. The benchmark may be associated with an assessment score indicative of a cognitive state prediction.

### CROSS-REFERENCE TO RELATED APPLICATIONS

This application is the US National Phase of International Application No. PCT/US2023/012153, filed Feb. 1, 2023, titled, DEMENTIA-RELATED NEURODEGENERATION TRACKING USING MAGNETIC RESONANCE IMAGING (MRI), which claims priority to Provisional Application No. 63,306,760, filed on Feb. 4, 2022; and of Provisional Application No. 63/407,575, filed Feb. 4, 2022, the disclosures of all of which are incorporate by reference herein in their entirety as if fully set forth herein.

### TECHNICAL FIELD

The invention relates to neurodegeneration biomarkers, and more particularly to neurodegeneration biomarker tracking based on imaging.

### BACKGROUND

The prevalence of Alzheimer's disease (AD) is increasing with the aging population. According to the World Health Organization, there are an estimated 36 million patients with AD in 2021 with approximately 6.5 million new cases reported annually (WHO, 2021). This is a progressive neurodegenerative disease characterized by cognitive decline and early atrophy within the medial temporal lobes (MTL) followed by progression to the rest of the brain.

Neurodegeneration often precedes cognitive decline, making atrophy assessed with structural magnetic resonance imaging (sMRI) an ideal clinical biomarker for detecting AD early and forecasting future cognitive and functional deterioration. Detecting AD prior to the point of irreversible neurodegeneration could improve the efficacy of currently available treatments (Coupé et al., 2015a) and improvements to current quantitative sMRI would add value in both clinical and research settings.

At later stages of AD, additional and validated quantitative sMRI metrics could add clinical utility while monitoring disease progression. Currently, the standard metric for this is neurocognitive assessment; however, neurocognitive assessments become more burdensome and less valuable as patients convert to moderate and severe AD. Regardless, there is still high clinical value in tracking a patient's disease progression and anticipating future deterioration throughout all stages of illness. sMRI is a passive, relatively short and inexpensive measurement, making it an ideal modality for monitoring disease progression in general, but especially in later stage AD patients.

sMRI is a well-validated tool for approximating diagnosis and disease progression in AD (Kantarci & Jack, 2003; Killiany et al., 2000). As such, sMRI has wide adoption in both clinical and research settings. The majority of the sMRI literature centers around hippocampal or other MTL measures. The magnitude of hippocampal volume atrophy is strongly associated with cognition, diagnosis, and AD-etiology (Csernansky et al., 2004; Gosche et al., 2002; Jack et al., 2002; Silbert et al., 2003). Hippocampal shape (Achterberg et al., 2014), texture (Sørensen et al., 2016), grading (Coupé et al., 2015b) and subfield volumes (Khan et al., 2015) are all validated hippocampus-based markers of AD-related etiology. The MTL has generally been a focus due of its critical role in memory and because these changes in volume are often detectable in cognitively normal (CN) individuals before symptoms of cognitive decline arise (REFS).

However, other parts of the brain provide important information, especially in the differential diagnosis of mild cognitive impairment (MCI), AD and frontotemporal lobe dementia (FTD), which have overlapping atrophy within the MTL (Rabinovici et al., 2007). A large body of research on AD-related etiology shows volume loss (Rabinovici et al., 2007) that quickly spreads (Braak et al., 1997; Dickerson et al., 2001; Frisoni et al., 1999; Gómez-Isla et al., 1996b; Laakso et al., 1996, 1998; Rabinovici et al., 2007; Thompson et al., 2003, 2007; Vercelletto et al., 2002) to the parietal lobe (Jacobs et al., 2012b), where gray and white matter loss likely occurs relatively early in cortical progression and is linked functionally to important AD symptoms such as impaired memory retrieval, naming ability, attention, and executive function (Cabeza et al., 2008; Jacobs et al., 2012a; Lindeboom & Weinstein, 2004). Over time and with variability in the order and rate of decline amongst patients, neurodegeneration spreads to posterior temporal, lateral occipital (Rabinovici et al., 2007), and left frontal lobes as well as limbic structures including the thalamus, cingulate gyrus, and nucleus accumbens (Nie et al., 2017). Because the underlying pattern of atrophy is important to consider in each patient, clinical imaging reports that quantify regional atrophy are standard in many dementia clinics and regional volumes are common endpoints in clinical research.

Several medical imaging companies have implemented regional brain volume reports in clinical settings. These tools are helpful in understanding the overall pattern of neurodegeneration. However, the spatiotemporal pattern of atrophy varies widely in patients, and it is difficult to quickly assess a patient's overall neurodegeneration from several numbers, reducing utility in diagnosis, which is most commonly done using neurocognitive assessment. Further, patients often receive scans at different facilities or on different scanners, and many of the current commercial tools do not account for these differences, affecting the validity of longitudinal follow-up using reports. A valid, harmonized, summary score with norms and cutoffs would be an easy addition to existing, clinically-implemented regional brain volume reports. Similarly, in research, several regions are frequently used as endpoints (i.e., hippocampal, entorhinal cortical, lateral ventricle, and whole brain volumes). Depending on the design and sample size of the study, reducing the number of tests could enhance sensitivity. Additionally, a validated, harmonized score that could be compared across multiple research studies could help researchers build on each other's findings by, for example, improving review articles and metanalyses. In both settings, a single score that summarizes AD-related neurodegeneration could add value.

Because atrophy patterns are complex and vary by patient, making the qualitative interpretation of several individual region of interest (ROI) results laborious and error-prone, researchers have made great efforts to condense sMRI information into a validated index for AD-related neurodegeneration. Early sMRI-based, multivariate approaches have focused on the classification of CN and AD (Casanova et al., 2018; Diciotti et al., 2012a). As the field has matured, approaches have aimed to classify more disease stages (i.e. CN. MCI, and AD) (Ma et al., 2019; Rallabandi et al., 2020) as well as predict who will stabilize and who will decline (Coupé et al., 2015c; Ma et al., 2019; Popuri et al., 2020). Most of these multivariate scoring methods utilize machine learning (Casanova et al., 2018; Diciotti et al., 2012a; Ma et al., 2019) and several combine sMRI with other imaging, biochemical, or clinical features to improve performance (Dukart et al., 2013a; Salvatore et al., 2018; Vemuri et al., 2009).

Noninvasive biomarkers of neurodegeneration, including blood and neuroimaging-based biomarkers, may also be indicative of early detection of dementia, disease severity, disease progression, and testing related to the efficacy of therapeutic interventions using a single number [1]-[3]. Improvements in biomarker identification and tracking are needed to assist with monitoring disease progression and benchmarking severity, among other things.

### DESCRIPTION

The objective of this study is to create and begin to validate a clinically meaningful and interpretable biomarker, called an “ADNeuro-Score” by summarizing AD-related neurodegeneration using only sMRI-based measurements. The approach differs from others through the development of a calculated value that uses a vector of multiple sMRI-based ROI-based features that are associated with cognitive decline. ROI-based features were selected because they are widely adopted by researchers and clinicians and because their significance is well understood. Several univariate, ROI-based approaches are similarly interpretable (REFS), however they do not capture a significant portion of sMRI features. There are several multivariate models that use machine learning approaches to generate probability scores (Casanova et al., 2018; Lu et al., 2021). While these types of black box models have been widely adopted for low stakes decisions such as online advertising, they have not yet been readily implemented in clinical settings where interpreting the results for each patient is important to providing care. Because the medical community has been slow to adopt these types of algorithms, a summary score was developed based on a calculated value using only well-understood sMRI features. The ADNeuro-Score is also computationally inexpensive, which may enable widespread research and clinical deployment. A creation of a single score, where probabilities of diagnosis or decline based on norms and cutoffs, can be used to monitor disease progression at all stages of the disease and compared across multiple sites, with established validity using either a common research tool like FreeSurfer (REF) or the widely available, FDA-cleared brain volumetric software package, Neuroreader® (Ahdidan et al., 2016).

The present invention presents systems and methods for tracking neurodegeneration and assessing cognitive state. Implementations of the present invention can provide and validate one or more clinically meaningful biomarkers related to neurodegeneration, e.g., an ADNeuro-Score.

Systems and methods may include at least one computing device configurated to receive a set of magnetic resonance (MR) images related to a brain region, determine from the MR images the volume of brain regions associated with cognitive decline, selected using cohort data, determine a benchmark based on a biomarker metric for a neurodegenerative disease, wherein the biomarker metric is validated in a cohort of data, and generate an assessment score that is harmonized for factors such as scanner features, age, and gender using a cohort of data, to predict a cognitive state.

In implementations, the brain regions include at least one of a cortical region and a sub-cortical region. The benchmark metric (biomarker) may be an adjusted hippocampal volume (AHV). The cognitive state may be at least one of cognitively normal (CN), mild cognitive impairment (MCI), Alzheimer's Disease (AD), or Dementia. A severity level may be determined based on a marker of cognition (a second biomarker) such as the Mini Mental Status Exam, the Alzheimer's Disease Assessment Scale-Cognitive Subscale, when the cognitive state is indicative of Alzheimer's Disease (AD).

As discussed herein, the cohort of data can include a set of demographic data, such as data related to at least one of a class, an age, a gender, and an education level. The benchmark determination may include an application of a machine learning algorithm (e.g., a machine learning algorithm trained on associations between biomarker metrics and neurodegenerative disease states, trained on identifying brain regions, cognitive states, and the like from MR images) and may further include application of a distance metric to the biomarker metric and cohort of data. The distance metric may be a Euclidean, Hausdorff metric and/or a Frechet distance. In various examples, the distance metric is computed based on a Z-weighted Euclidean distance (ZWE), according to:

$$ZWE = {{\sum }_{i = 1}^{NR} [ ( {w}_{i}^{T} - {w}_{i}^{S} ) {z}_{i} ]}^{2} ,$$

where zᵢ refers to an average of z-transformed p-value across possible pairwise comparisons for a given region, i, and NR denotes the total number of regions. The possible pairwise comparisons include cognitively normal versus mild cognitive impairment, cognitively normal versus Alzheimer's Disease, and mild cognitive impairment versus Alzheimer's Disease.

The scope of the invention also includes computer-readable media and/or at least one computing system including a processor that executes stored instructions for executing the steps of the method. The above and other characteristic features of the invention will be apparent from the following detailed description of the invention.

### BRIEF DESCRIPTION OF THE DRAWINGS

The drawings illustrate generally, by way of example, but not by way of limitation, various aspects discussed in the present document. The above and other objects and advantages of the invention will be apparent to those skilled in the art based on the following detailed description in conjunction with the appended figures, of which:

FIG. 1 illustrates a flowchart for predicting a cognitive state in accordance with implementations.

FIG. 2 illustrates a table comprising cohort data in accordance with implementations.

FIG. 3 illustrates a flowchart visualizing baseline allocation of subjects into the 3 experimental cohorts (ROI Selection, CN Template, and Experimental).

FIG. 4 illustrates various regions of interest and z-scored-based weighting, in accordance with implementations discussed herein.

FIG. 5 illustrates baseline classification performance of the AD-Neuro score and AHV across various diagnostic groups.

FIG. 6 illustrates violin plots depicting the distribution of ADNeuro-Score and AHV for various diagnostic groups.

FIG. 7 illustrates ADNeuro-Score and AHV correlations, in accordance with implementations discussed herein.

FIG. 8 illustrates a diagram of an exemplary computer system, in accordance with implementations discussed herein.

FIG. 9 illustrates a machine learning and training model in accordance with implementations discussed herein.

### ADDITIONAL DESCRIPTION OF ILLUSTRATIVE IMPLEMENTATIONS

The present disclosure may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular implementations by way of example only and is not intended to be limiting of the claimed invention.

The present invention presents systems and methods for predicting a cognitive state in accordance with implementations. Implementations of the present invention can provide and validate one or more clinically meaningful biomarkers related to neurodegeneration, e.g., an ADNeuro-Score. Implementations can utilize structural Magnetic Resonance Imaging (sMRI) techniques to approximate disease diagnosis and progression for Alzheimer's Disease-related neurodegeneration. Such techniques can help consolidate and understand complex atrophy patterns, which can vary by patient. In various implementations, a clinically meaningful and interpretable metric can summarize AD-related neurodegeneration from a single, T1-weighted MRI scan.

A major focus in Alzheimer's disease (AD) research is on discovering noninvasive biomarkers that can detect dementia early, benchmark disease severity, monitor disease progression, predict worsening, and aid in testing the efficacy of therapeutic interventions. Structural Magnetic Resonance Imaging (sMRI) is a well-validated, widely adopted, in-vivo measure of underlying neuropathology. For these reasons, researchers have made great efforts to condense sMRI information into a validated index for AD-related neurodegeneration. Implementations describe an algorithm to calculate an “ADNeuro-Score,” using regional volume measurements associated with cognitive decline.

ADNeuro-Score computations utilize a novel modified Euclidean-inspired distance function to calculate the differences between each participant and a cognitively normal older adult template, adjusting for intracranial volume, age, sex, and scanner model. Examples discussed herein report validation results to assess sensitivity to diagnoses, e.g., cognitively normal (CN), mild cognitive impairment (MCI), and AD) and disease severity (CDR-S, MMSE, and ADAS-11), in 929 older adults (mean age=72.67 years) drawn from the Alzheimer's Disease Neuroimaging Initiative (ADNI) study. The relationship between ADNeuro-Score and diagnosis was evaluated using pairwise, two-tailed t-tests. A linear model assessed associations between ADNeuro-Score and disease severity scores. Z-tests on the Fisher z-transformed correlation coefficients were performed to determine if ADNeuro-Score performance was significantly different from AHV. An alpha=0.05, Bonferroni corrected was used in analyses.

In order to determine if ADNeuro-Score might be predictive of disease progression, the relationship between ADNeuro-Score at baseline and both diagnostic transition category (DTC) (Diagnosisstable or Diagnosisdecline) and change in disease severity at 12-, 24-, 36-, and 48 months were examined. Sub-analyses were performed, which binned participants based on a diagnosis at baseline (e.g., CN or MCI). ADNeuro-Score was found to be significantly sensitive to diagnosis, and performed as well as the benchmark, AHV.

Further, ADNeuro-Score was significantly associated with all disease severity scores at baseline. Associations between ADNeuro-Score and disease severity (CDR-SB and ADAS-11) and were significantly stronger than with AHV. The relationship between ADNeuro-Score at baseline and DTC was significant at all time points and performance was equivalent to AHV. In individuals with MCI at baseline, there were significant effects of DTC in ADNeuro-Score value at all timepoints and equivalent performance using AHV. There were weak to no significant effects in CN individuals. AHV performed somewhat better than ADNeuro-Score at distinguishing DTC in this group. Significant associations were found between ADNeuro-Score at baseline and change in disease severity scores at all time points. Correlations between ADNeuro-Score and disease severity (ADAS-11) at 24 months was significantly stronger than with AHV, but otherwise equivalent to the benchmark. This was also driven largely by individuals with a diagnosis of MCI at baseline.

These early validation results suggest that ADNeuro-Score has potential to be a clinically meaningful biomarker for dementia. It performs as well or better than the benchmark, AHV. It is also an easy to interpret and computationally cheap, which improves ease of deployment and incorporation into existing research and clinical workflows. ADNeuro-Score utilizes more sMRI information than a single region while still focusing on AD-affected structures. For these reasons, future directions of this research may include determining if this novel, calculated metric will add value in tracking disease progression at later stages of AD and in the differential diagnosis of dementia.

### Abbreviations

Alzheimer's Disease (AD)
Alzheimer's Disease Assessment Scale-Cognitive Subscale (ADAS-Cog)
Amnestic Mild Cognitive Impairment (aMCI)
Area Under the Receiver Operator Characteristic Curve (AUC-ROC)
Clinical Dementia Rating Scale Sum of Boxes (CDR-SB)
Medial Temporal Lobe (MTL)
Mild Cognitive Impairment (MCI)
Mini Mental State Exam (MMSE)
Protected Health Information (PHI)
Receiver Operator Characteristic (ROC)
Region of Interest (ROI)
Standard Deviation (SD)
Structural Magnetic Resonance imaging (sMRI)

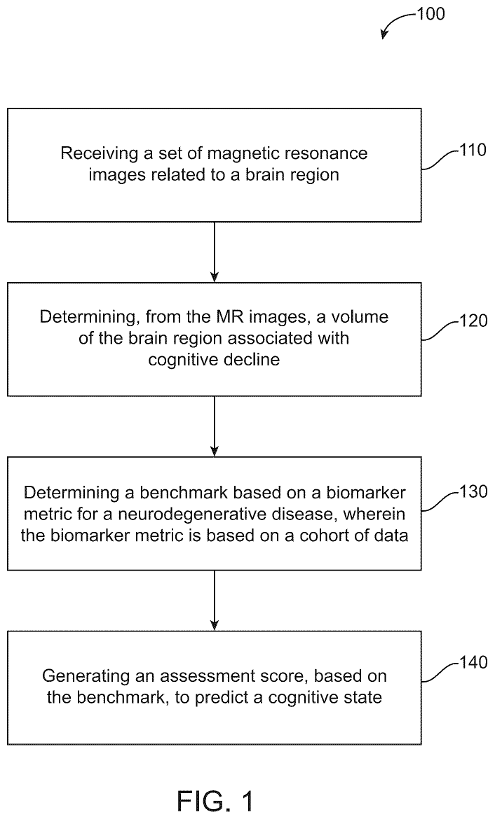

FIG. 1 illustrates a flowchart for predicting a cognitive state based on biomarker tracking, using magnetic resonance imaging data. In various implementations, magnetic resonance images indicative of a brain region may be received 110, e.g., by a computing system. Brain regions may include a cortical region, a sub-cortical region, and/or a brain region associated with the disease of interest. MR images can illustrate slices of a brain, which comprises a plurality of regions. A volume of the brain regions from MR images sets may be associated with cognitive decline 120. Various image analysis techniques may be applied to the set of MR images to determine these potential areas of interest.

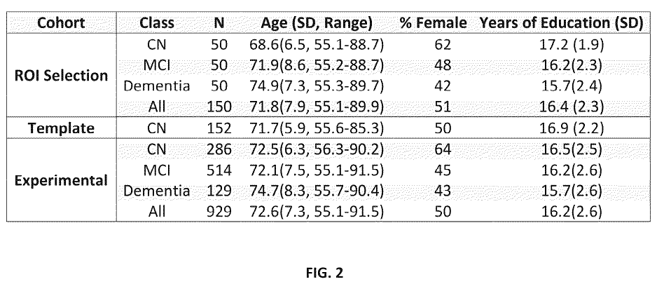

Data may be extracted from the brain images, to provide information about at least one biomarker metric. Each biomarker metric may be associated a neurodegenerative disease, such as Alzheimer's Disease, among others. Biomarkers metrics, as discussed herein, may further be associated with a cohort of data, as further described with respect to FIG. 2. Biomarker metrics may be applied to determine a benchmark 130, e.g., related to a disease state, disease progression, severity, and the like. Based on the benchmark, an assessment score may be determined to predict a cognitive state 140. In examples, the cognitive state is at least one of a cognitively normal (CN) state, a mild cognitive impairment (MCI), or Alzheimer's Disease (AD). In various implementations, the biomarker metric may be adjusted hippocampal volume (AHV).

An assessment score, which may be determined from the methods discussed herein, predicts a cognitive state. When a neurodegenerative disease, such as AD, is identified for example, a second biomarker may be analyzed to determine a severity level. Other biomarkers that may be applied may relate to disease progression, disease type, and the like.

FIG. 2 illustrates a table comprising data cohorts, as discussed herein. In examples, a cohort can comprise a set of demographic data related to one or more categories. A cohort can represent, for example, a collection of data related to at least one of a region of interest (ROI), a template dataset, or an experimental dataset. The template dataset may be indicative of known and/or recorded values. The experimental dataset can include information related to the individuals and/or images of interest.

The demographic data may comprise a class, a number of individuals and/or data points (N), an age, a gender, gender percentage, and a education level, such as a number of years of education. For each of the demographic data categories, a standard deviation and/or range may be provided. Class demographic data may be indicative of a cognitive state, such as cognitively normal, mild cognitive impairment, dementia, or a combination of one or more classes. (N) may be indicative of a number of data points within the category. Age may be represented as average age (with a range and standard deviation), gender may be represented by a % Female or % Male, and education may provided by an average years of education, with a standard deviation.

One or more data cohorts may provide biomarker metrics to assist with the determination of a benchmark. In various examples, a distance metric, such as a Hausdorff metric or a Frechet distance, may estimate differences between individual and average metrics to compute the benchmark and generate an assessment score, such as an ADNeuro-Score as discussed herein. One or more machine learning algorithms may be applied to generate benchmarks and compute assessment scores, based on one or more cohorts of data. For example, the machine learning algorithm may be trained on associations between biomarker metrics and neurodegenerative disease states.

### ADNeuro-Score Example 1

The following examples apply aspects of the present invention to generate an assessment score, referred to as ADNeuro-Score. In the examples, data sets from cognitively normal (CN) individuals and patients with a mild cognitive impairment (MCI) or dementia diagnosis were drawn from the Alzheimer's Disease Neuroimaging Initiative (ADNI) [4].

### Methodology:

Eighty-four cortical and subcortical regional volumes were estimated from T1-weighted MR images using FreeSurfer [5]. To determine which regional volumes were associated with cognitive decline, an ANOVA with an alpha=0.05, Bonferroni corrected, was performed on a random selection of 150 participants (N=50 each CN, MCI, and dementia). A vector containing the resulting 41 regions was extracted for experimental and template cohorts, as illustrated in FIG. 2. These regions were consistent with the existing literature [6], [7], [8].

To compute ADNeuro-Score, a Hausdorff distance metric was used to estimate the differences between individual and average template vectors. ADNeuro-Score was benchmarked using adjusted hippocampal volume (AHV), which is a NIA-AA diagnostic biomarker for Alzheimer's disease [9], the most common form of dementia [10]. Both ADNeuro-Score and AHV were harmonized for intracranial volume, age, sex, and scanner model [11]. Validation used an experimental cohort (N=929, mean age=72.67 years) and tested sensitivity to diagnosis using pairwise t-tests and an alpha=0.001, Bonferroni corrected. Results were converted to a z-score. Additional association testing was performed with respect to disease severity, operationalized as mini-mental state exam (MMSE) and Alzheimer's Disease Assessment Scale-Cognitive Subscale (ADAS-Cog) scores), using at least one linear regression model.

Results: Both ADNeuro-Score and AHV differed between all three cognitive groups. ADNeuro-Score (z=10.0) better differentiated MCI from dementia than AHV (z=8.9). AHV (z=7.2) was slightly better at distinguishing MCI from CN than ADNeuro-Score (z=6.6). Both ADNeuro-Score and AHV were similarly associated with MMSE (RADNS=0.41; RHA=0.41) and ADAS-Cog (RADNS=0.49; RHA=0.47) scores.

Accordingly, the ADNeuro-Score provided an improved, robust and reliable way to distinguish CN, MCI and AD patients. The ADNeuro-Score assessment performed equivalently to AHV in predicting MMSE and ADAS-Cog assessment scores. Additional applications of the present implementations can apply ADNeuro-Score with respect to monitoring disease progression and improving the differential diagnosis of AD.

### ADNeuro-Score Example 2

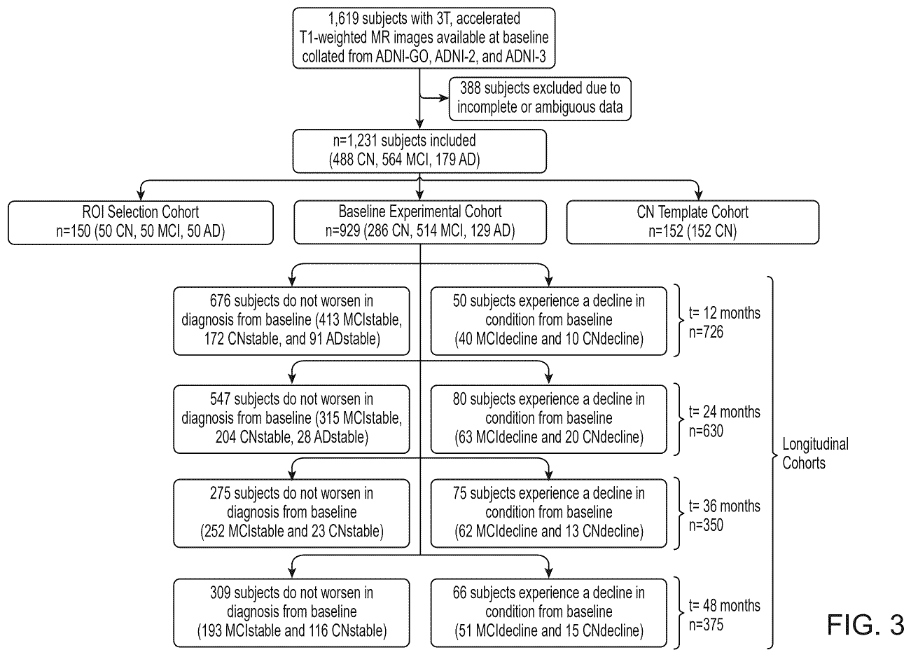

FIG. 3 illustrates a flowchart visualizing baseline allocation of subjects into the three experimental cohorts (Region of Interest Selection, CN Template, and Experimental) and longitudinal construction of the stable and decline groups at 12, 24, 36, and 48-months. Patients whose diagnosis progressed relative to baseline were classified into a Diagnosis (decline) group, while those who did not worsen were categorized as Diagnosis (stable) for each respective time point.

In the cohorts, cognitively normal individuals and patients with a mild cognitive impairment (MCI) or mile AD dementia diagnosis were drawn from the Alzheimer's Disease Neuroimaging Initiative (ADNI). In an example, eighty-four cortical and subcortical regional volumes were estimated from T1-weighted MR images.

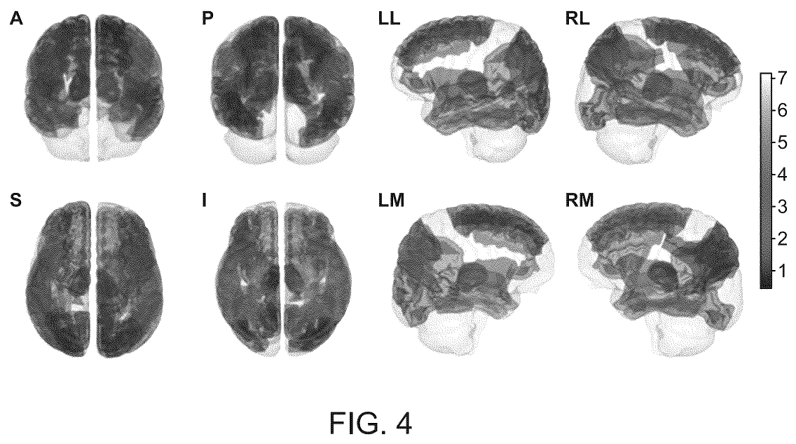

To determine which regional volumes are significantly associated with cognitive decline, a random module (e.g., NumPy random module) can pseudo-randomly separate age- and sex-matched participants into an ROI Selection Cohort (Table 1, below; FIG. 3) and performed an ANOVA for each of the 84 ROIs with an alpha=0.05, Bonferroni corrected (FIG. 4)

From the remaining participant pool, a sample of CN individuals were pseudo-randomly selected to generate a CN template representative of the healthy average brain (See CN Template Cohort), and the remaining participants were allocated to the Experimental Cohort (Table 1 below; FIG. 4)

Table 1. Demographic Information Full description of all cohort demographics at baseline. Total number of subjects (n), sex (by percentage female), age and education in years are reported for each cohort in full (All), as well as for CN, MCI, and AD diagnostic subdivisions. The CN Template Cohort was comprised of only CN individuals. Values for age and education are summarized in the form mean (±standard deviation, min-max) and mean (±standard deviation), respectively.

| | Clinical | Group | | | |
|---|---|---|---|---|---|
| Dataset | Measure | CN | MCI | AD | All |
| ROI | n | 50 | 50 | 50 | 150 |
| Selection | Subjects (% | 62 | 48 | 42 | 51 |
| | Female) | | | | |
| | Age | 68.6 | 71.9 | 74.9 | 71.8 |
| | | (±6.5, 55.1-88.7) | (±8.6, 55.2-88.7) | (±7.3, 55.3-89.7) | (±7.9, 55.1-89.9) |
| | Education | 17.2 (±1.9) | 16.2 (±2.3) | 15.7 (±2.4) | 16.4 (±2.3) |
| Template | n | 152 | | | |
| | Subjects (% | 50 | | | |
| | Female) Age | 71.7 | | | |
| | Education | (±5.9, 55.6-85.3) | | | |
| | | 16.9 (±2.2) | | | |
| Experimental | n | 286 | 514 | 129 | 929 |
| | Subjects (% | 64 | 45 | 43 | 50 |
| | Female) | | | | |
| | Age | 72.5 | 72.1 | 74.7 | 72.6 |
| | | (±6.3, 56.3-90.2) | (±7.5, 55.1-91.5) | (+8.3, 55.7-90.4) | (±7.3, 55.1-91.5) |
| | Education | 16.5 (±2.5) | 16.2 (±2.6) | 15.7 (+2.6) | 16.2(±2.6) |

FIG. 4 illustrates various regions of interest and z-scored-based weighting, in accordance with implementations discussed herein. The 41 significant regions of interest (ROIs) extracted by performing ANOVA for each of the 84 regions in the ROI selection cohort are visualized above on the Allen 500-micron Human Brain Atlas. The gray scale depicts the z-score-based weighting of each ROI in ADNeuro-Score (ADNS), as described herein. The following letters denote anatomical orientation: Anterior (A), Posterior (P), Superior(S), Inferior (I), Left Lateral Surface (LL), Left Medial Surface (LM), Right Lateral Surface (RL), Right Medial Surface (RM).

### Methodology:

In implementations, a vector containing the 41 significant ROIs were extracted for experimental and CN template cohorts. Data was adjusted to account for confounding covariates (age, sex, intracranial volume, and scanner model) by modeling the raw structural volume of each ROI as the linear combination of all covariates with multiple linear regression, plus a residual, and then taking the z-transformation of the residual term (also known as the standardized residual or w-score)

To compute an ADNeuro-Score, a modified Euclidean inspired distance function, referred to herein, as the Z-weighted Euclidean distance (ZWE), was used to estimate the differences between individual and average template vectors. The ZWE distance function was computed by applying a weight drawn from each regions' level of significance determined in the ROI selection process, as shown in Equation (1):

$$ZWE = {{\sum }_{i = 1}^{NR} [ ( {w}_{i}^{T} - {w}_{i}^{S} ) {z}_{i} ]}^{2} \qquad ( Eq . 1 )$$

wherein, zᵢ refers to the average of the z-transformed p-value across the 3 possible pairwise comparisons (CN vs MCI, CN vs AD, and MCI vs AD) for a given region, I, and NR denotes the total number of regions.

The ZWE distance was computed between the harmonized residual of each subject and that of the CN template. The ADNeuro-Score was benchmarked using adjusted hippocampal volume (AHV), which is an NIA-AA diagnostic biomarker for Alzheimer's Disease, the most common form of dementia.

### Results:

Sensitivity to diagnosis was assessed by calculating area under the ROC curve (AUC-ROC) and 95% confidence intervals for each diagnostic group comparison (Table 2). A sensitivity to diagnosis was further tested using pairwise, two-tailed t-tests for each possible diagnostic group comparison with an alpha=0.001, Bonferroni corrected (Table 2). Results were then converted to z-scores and Cohen's D was calculated to indicate effect size. An association with disease severity, operationalized as MMSE, ADAS-11, and CDR-SB scores, was also tested using linear regression in the experimental cohort (Table 2).

In addition, a t-distribution tested the null hypothesis that the coefficient (or slope) of each line is equal to zero. A z-test performed on the Fisher z-transformed correlation coefficients determined whether one metric's performance was statistically significantly different from another.

### Validation:

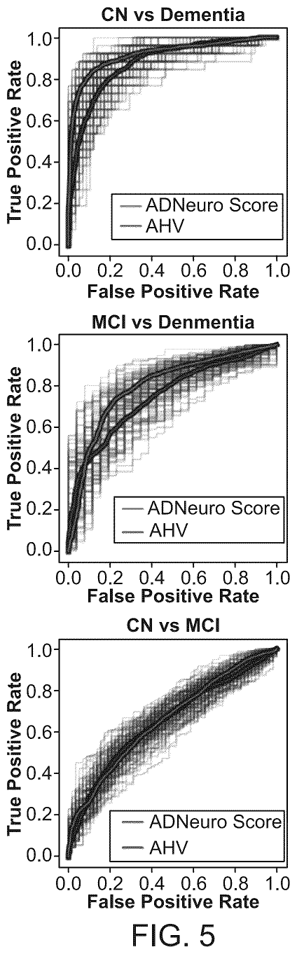

Across all diagnostic group comparisons, ADNeuro-Score performed as well as AHV (p<0.001 for both metrics across all comparisons; Table 2). The largest effect size for both metrics was found for the CN-AD group comparison, and the smallest effect sizes were found between CN and MCI groups (Table 2). Although ADNeuro-Score and AHV demonstrate similar AUC-ROC values across all group comparisons (Table 2), the overlaid ROC curves for the two metrics indicate that for the two AD comparisons (CN vs AD and MCI vs AD), particularly at low false positive rates, the true positive rate of ADNeuro-Score tends to surpass that of AHV (FIG. 5).

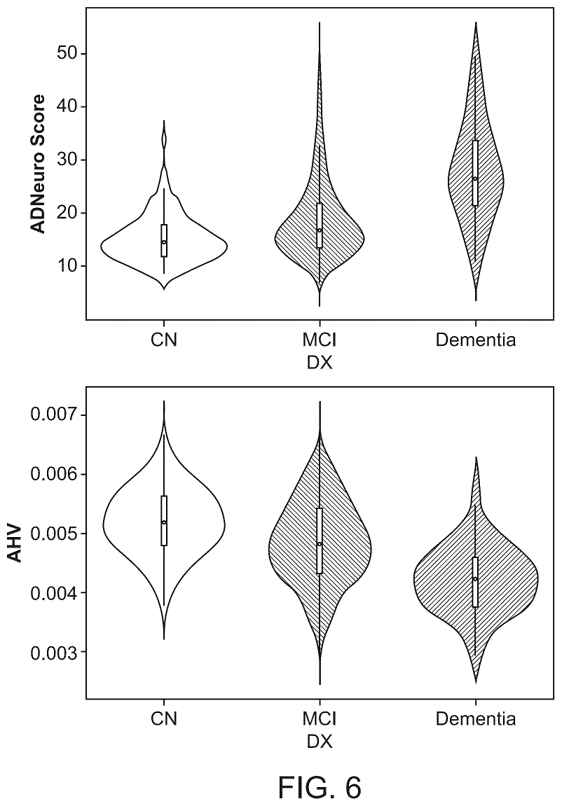

FIG. 6 illustrates the distribution of AHV and ADNeuro-Score for each diagnostic group at baseline as violin plots. Notably, ADNeuro-Score demonstrates greater separation between the diagnostic class medians with a more central concentrated distribution and longer tails relative to AHV. The ADNeuro-Score and AHV were equivalently correlated with MMSE, ADAS-11, and CDR-SB scores, as measured by correlation coefficient magnitude (Table 2). Each of these correlations had a slope that is statistically significantly different than zero for both metrics (p<0.001).

Z-tests on the Fisher z-transformed correlation coefficients found that the respective correlations between ADNeuro-Score and CDR-SB and ADAS-11 were significantly stronger than those between the same neuropsychological scale scores and AHV (p=0.006 for CDR-SB and p=0.024 for ADAS-11). Metric correlations stratified by diagnostic group revealed the most pronounced difference in biomarker performance in the AD group (Table 4). In the AD sub-analysis, the correlations between ADNeuro-Score and ADAS-11 and CDR-SB were found to be statistically significant (p<0.001 for ADAS-11 and p<0.01 for CDR-SB), while none of the AHV correlations were found to be significant.

Table 2. Metric Sensitivity to Diagnosis Results: ADNeuro-Score benchmarking against adjusted hippocampal volume (AHV) based on metric sensitivity to diagnosis, operationalized as pairwise, two-tailed t-tests performed for each possible diagnostic group comparison (cognitively normal; CN, mild cognitive impairment; MCI, Alzheimer's Disease; AD), z-scores, and effect sizes (Cohen's d). For each group comparison at baseline, AUC values and 95% confidence intervals are included. Significant group comparisons are denoted by * to indicate p<0.05, ** to indicate p<0.01, and *** to indicate p<0.001.

| Group | | | | z- |
|---|---|---|---|---|
| Comparison | Metric | AUC [95% CI] | Cohen's d [95% CI] | score |
| CN vs MCI | ADNS | 0.66 [0.59, 0.73] | −0.56 [−0.59, −0.53]*** | −7.46 |
| | AHV | 0.65 [0.58, 0.72] | 0.55 [0.52, 0.58]*** | −7.27 |
| CN vs AD | ADNS | 0.91 [0.86, 0.96] | −2.06 [−2.24, −1.88]*** | −16.37 |
| | AHV | 0.88 [0.81, 0.95] | 1.72 [1.55, 1.89]*** | −14.27 |
| MCI vs AD | ADNS | 0.80 [0.71, 0.89] | −1.16 [−1.19, −1.14]*** | −11.23 |
| | AHV | 0.76 [0.67, 0.84] | 0.92 [0.90, 0.94]*** | −9.04 |

### Conclusion

In this early validation experiment, ADNeuro-Score indicted significant sensitivity to diagnoses (e.g., CN, MCI, and AD); significant association with disease severity (e.g., MMSE, ADAS-11, and CDR-SB); and comparable performance with the benchmark, AHV, across all assessments. Future directions include determining if ADNeuro-Score can improve the differential diagnosis of dementia and/or disease progression tracking, particularly in later stages of Alzheimer's Disease.

### ADNeuro-Score Example 3

### Methodology:

### Data Acquisition and Demographics

CN individuals and patients with MCI or AD diagnosis were drawn from the Alzheimer's Disease Neuroimaging Initiative (ADNI) database (adni.loni.us.edu) (Jack et al., 2008; Mueller et al., 2005b, 2005a). The ADNI is a global research study launched in 2003, primarily aimed at evaluating biological markers to measure the progression of MCI and early AD. Biomarkers will be derived from MRI, PET, and blood and CSF-based biospecimen assays and their discovery and validation are intended to facilitate the development of treatments to slow or halt AD progression.

ADNI studies were conducted according to the Good Clinical Practice guidelines, the Declaration of Helsinki, the U.S. 45 Code of Federal Regulations (CFR) Part 46 and 21 CFR Part 50-Protection of Human Subjects, and 21 CFR Part 56-Institutional Review Boards. Written informed consent and HIPAA authorizations were obtained from all participants or authorized representatives prior to the conducting of protocol-specific procedures. The ADNI protocol was approved by the Institutional Review Boards of all participating institutions, listed in File 1 in Supplementary Materials (Mukherji et al., 2021).

In this work, a total of 1,619 subjects with available 3T, accelerated, T1-weighted MR images at baseline were collated from the ADNI-GO, ADNI-2, and ADNI-3 study phases; of these, 388 subjects were discarded due to incomplete or ambiguous data. Acquisition methods are detailed by Chow et al. (Chow et al., 2015). The remaining cohort of 1,231 individuals was comprised of 488 CN, 564 MCI, and 179 mild AD. Participants were diagnosed at baseline and reassessed at each study visits (Petersen et al., 2010). The study sample was subdivided into the following three cohorts: region of interest (ROI) selection, cognitively normal template creation, and experimental (see Table 1 for a full description of all cohort demographics).

### Region of Interest Selection Cohort

To determine which regional volumes were associated with cognitive decline, a sample of 150 age- and sex-matched individuals from the overall participant pool were pseudo-randomly selected using the numpy random module (Harris et al., 2020) with equivalent proportions of each diagnostic category (see Table 1 for a full description of ROI selection cohort demographics) and grouped into a cohort in order to quantitatively determine ROI inclusion and weighting (see Methods: ROI Selection).

### Cognitively Normal Template Cohort

A sample of cognitively normal, age- and sex-matched individuals were pseudo-randomly randomly selected from the remaining participant pool to generate a CN template. The purpose of this template was to represent the average, healthy older adult brain (see Table 3 for a full description of CN template demographics). The sample size for this cohort was set to 152 individuals, to match that which was drawn from a normative adult population to construct the MNI152 template, a ubiquitous structural brain atlas based on the group-wise registration of 152 3D, T1-weighted MR images (Mazziotta et al., 1995a, 1995b).

### Experimental Cohort

The remaining 929 participants were grouped into an experimental cohort for sMRI metric extraction and testing (see Table 3 for a full description of experimental cohort demographics). Baseline analyses were conducted for all 929 participants of the experimental cohort.

Longitudinal analyses were conducted on a subset of participants with available follow-up session data and diagnosis. These participants were further divided into diagnostic transition category (DTC), where Diagnosisstable or Diagnosisdecline groups were based on their diagnosis at 12-, 24-, 36- and 48-month follow-up sessions. The Diagnosisdecline groups were defined by a worsening of diagnosis. Those who transitioned from CN to MCI or CN to AD were denoted as CNdecline. Those who transitioned from MCI to AD were denoted as MCIdecline. There was no later stage diagnostic category than AD in this sample, and therefore no ADdecline group. The Diagnosisstable group was defined by an unchanging diagnosis or, in rare cases, an improved diagnosis. These were referred to as MCIstable, CNstable, and ADstable (see FIG. 1 for a flowchart detailing the construction of longitudinal cohorts).

### Neurocognitive Assessments

Participant scores on the Clinical Dementia Rating Scale Sum of Boxes (CDR-SB), Alzheimer's Disease Assessment Scale Cognitive Subscale (ADAS-11), and Mini-Mental State Exam (MMSE) were collated for all experimental cohort participants with scores available at each time point. The original ADAS-Cog (Rosen et al., 1984), referred to as the ADAS-11, is comprised of 11 subtests whose scores are summed (unweighted) to generate a total raw score from 0 to 70. Higher scores on the ADAS-11 correspond with a larger number of errors and poorer performance, as rated by clinicians (Grochowalski et al., 2015). The MMSE is short, 11 items questionnaire routinely used to measure cognitive impairment (Folstein et al., 1975). The MMSE has six subdomains: time orientation, place orientation, registration, attention, recall, language, and visual construction and yields a score from 0 to 30, with lower scores representing greater cognitive dysfunction The CDR-SB (Balsis et al., 2015; Hughes et al., 1982; Morris, 1993; O'Bryant et al., 2008) similarly measures cognitive function, along with functional ability, as rated by clinicians across six domains (also referred to as boxes): memory, orientation, judgement and problem-solving, community affairs, home and hobbies, and personal care. Individual scores for each domain (or box) are summed to produce a total score ranging from 0 to 18, where higher scores stage more severe dementia (O'Bryant et al., 2008).

### ADNeuro-Score

Cortical reconstruction and volumetric segmentation of the 3T, accelerated, T1-weighted MR images was performed with version 7.1 of the Freesurfer image analysis suite, which is documented and freely available for download online (http://surfer.nmr.mgh.harvard.edu/). The details of these procedures are described in previous publications (Dale et al., 1999; Desikan et al., 2006; Fischl et al., 2001, 2002; Fischl, Salat, et al., 2004; Fischl, Sereno, & Dale, 1999; Fischl, Sereno, Tootell, et al., 1999; Fischl, van der Kouwe, et al., 2004; Jovicich et al., 2006; Reuter et al., 2010; Segonne et al., 2004, 2007). With these methods, eighty-four cortical and subcortical regional volumes were estimated at baseline for each subject. Additionally, results were reviewed using the ENIGMA structural imaging quality control protocols (http://enigma.us.edu/) (Stein et al., 2012).

### ROI Selection

In order to determine which brain regions were most sensitive to diagnosis at baseline, a quantitative analysis was performed in the ROI selection cohort using an Analysis of Variance (ANOVA) to test for an effect of diagnosis for each of the 84 regions with an alpha=0.05, Bonferroni corrected. The resulting 41 significant brain regions were used to compute ADNeuro-Score (Table 3) and were consistent with the existing literature (Harper et al., 2017; Yin et al., 2013; Zanchi et al., 2017).

### Cognitively Normal Template

To generate a CN template vector representative of the healthy average brain, each volume that survived the ROI selection process was averaged across all CN template cohort participants (see Table 2). Data harmonization procedures were applied to each region in the CN template vector (see Methods, Data Harmonization). This CN template vector had the same dimensions as vectors extracted from experimental cohort participants. These two vectors were used to compute the distance metrics evaluated in this work (see Methods, Determining the Optimal Distance Metric).

### Data Harmonization

Neuroimaging research has moved towards collecting large samples across multiple sites and scanners to facilitate a major expansion in sample size, allowing researchers to better detect effects and reduce the chances of making erroneous conclusions based on skewed, outlier-driven results. While these are significant benefits, multi-site and-scanner studies are susceptible to unwanted data variability driven by the effects of confounding covariates, such as scanner manufacturer and model, which reduces sensitivity to detecting effects (Alfaro-Almagro et al., 2021). Other common sources of variability in sMRI-derived brain volume analysis include differences in brain morphology and head size between participants (Voevodskaya, 2014; Whitwell et al., 2001).

A challenge of the present study was mitigating variability in the data introduced by factors such as interindividual variations in head size, age, sex, and MRI acquisition features like scanner model and manufacturer. To this end, a data harmonization procedure was employed to account for confounding covariates by applying a w-score, a harmonization approach previously validated on sMRI data (Ma et al., 2019; Popuri et al., 2020). Harmonization utilized a generalized linear model (GLM) framework, where the structural volume for a given region of each participant was modeled as the linear combination of all covariates as shown in Equation (2):

$${V}_{i}^{r} = {β}_{o}^{r} + {\sum }_{c}^{NC} {β}_{c}^{r} {x}_{c , i} + {ℰ}_{i}^{r} \qquad ( Eq . 2 )$$

Here, the volume

$${V}_{i}^{r}$$

of region r for subject i, is modeled as the sum over NC covariates (age, sex, scanner model, and total ICV) of coefficient

$${β}_{c}^{r} ,$$

for covariate c, multiplied by the value of the covariate, x_{c,i}, plus the residual term,

$${ℰ}_{i}^{r} .$$

To compute the w-score,

$${w}_{i}^{r} ,$$

also known as the standardized residual, the z-transformation of the residual term from Eq. (1),

$$ℰ {i}_{r}^{}$$

is taken, as shown in Equation (3):

$${w}_{i}^{r} = \frac{( {ℰ}_{i}^{r} - {μ}_{ℰ}^{r} )}{{σ}_{ℰ}^{r}} \qquad ( Eq . 3 )$$

where

$${μ}_{ℰ}^{r}$$

and

$${σ}_{ℰ}^{r}$$

represent the mean and standard deviation, respectively, of the residual term for a given subject's regional brain volume. In this way, regional volumes used to calculate ADNeuro-Score were adjusted for the effects of age, sex, head size, and scanner model.

### Calculating the Z-Weighted Euclidean Distance

To compute the difference between each experimental cohort participant and the CN template, a novel modified Euclidean inspired distance function, callws the Z-weighted Euclidean distance (ZWE) was employed. A traditional Euclidean distance metric is calculated by treating the list of significant, harmonized regional volumes as a vector in n-dimensional space, where n is the number of regions, and computing the Euclidean distance between each participant and the CN template vectors. The ZWE distance function differs only by multiplying a weight drawn from each region's level of significance, which was determined in the ROI selection process, as shown in Equation (4):

$$ZWE = {{\sum }_{i = 1}^{NR} [ ( {w}_{i}^{T} - {w}_{i}^{S} ) {z}_{i} ]}^{2} \qquad ( Eq . 4 )$$

where zᵢ refers to the average of the z-transformed p-value across the three possible pairwise comparisons (CN vs MCI, CN vs AD, and MCI vs AD) for a given region, i, and NR denotes the total number of regions. The ZWE distance was computed between the harmonized residual of each subject,

$${w}_{i}^{S} ,$$

and the CN template,

$${w}_{i}^{T} .$$

The significant KOIs listed in Table 2 are additionally visualized in FIG. 2, with a gray scale indicating respective zᵢ weighting.

Several other methods were examined for computing the difference between each experimental cohort subject and the CN template. Each of these algorithms similarly involved computing a distance function between like objects constructed from the harmonized, significant regional brain volumes, and determined in the ROI selection step. Generally, the distance functions fell into one of two categories (curves and points). For the distance functions that computed distances between curves, in addition to projecting the curve in one dimensional space, the k-false nearest neighbors method was implemented to embed each curve in n-dimensions. The functions which computed a distance between curves included the Fréchet distance and the Hausdorff distance. The Fréchet Distance is one method used to quantify the similarity between two curves or sets of points in space, with an emphasis on the location and ordering of points (Dumitrescu & Rote, 2004). Similarly, the Hausdorff distance measures how far two subspaces of a metric space are from each other; however, it does not account for the flow or order of points (Maiseli, 2021). The vector-based distance functions included the Euclidean and ZWE distance. Further details along with equations modeling these mathematical distances are included in Table 10.

Statistical analyses tested the performance of each distance metrics based on sensitivity to diagnosis, disease severity, and progression (See, e.g., Tables 7-10). All metrics evaluated were benchmarked using AHV, an NIA-AA diagnostic biomarker for Alzheimer's disease (Jack et al., 2018) (Table 7). The optimal biomarker, the ZWE distance, was selected as our “ADNeuro-Score.” (See Validation Procedures).

### Validation Procedures

### Baseline Validation Procedures

At baseline, sensitivity to diagnosis was evaluated using pairwise, two-tailed t-tests for each possible diagnostic group comparison (CN vs MCI, MCI vs AD, and CN vs AD), with an alpha=0.001, Bonferroni corrected (Table 4). Results were then converted to z-scores and Cohen's d was calculated to indicate effect size (Table 4). Sensitivity to diagnosis was further assessed by calculating area under the Receiver Operating Characteristic curve (AUC-ROC) and 95% confidence intervals for each diagnostic group comparison at baseline.

Baseline association with disease severity, operationalized as MMSE, ADAS-11, and CDR-SB scores, was also tested using linear regression, both in the overall baseline experimental cohort and in each diagnostic sub-group (Table 5). For all baseline and longitudinal linear regression results, p values were obtained by using the t-distribution to test the null hypothesis that the coefficient (or slope) of the line is equal to zero. Additionally, a z-test on the age z-transformed correlation coefficients was performed at all time points to determine if one metric's performance was statistically significantly different from another.

### Longitudinal Validation Procedures

In order to determine if ADNeuro-Score might be predictive of disease progression, the relationship between ADNeuro-Score at baseline and both diagnostic transition category (DTC) and disease severity was assessed at 12, 24, 36, and 48 months. The relationship between ADNeuro-Score at baseline and DTC was evaluated longitudinally with pairwise, two-tailed t-tests comparing the distribution of the metrics between patients whose diagnosis either worsened (Diagnosis_{decline}) or did not decline ₍Diagnosis_{stable)} from baseline, as indicated by diagnosis at 12-, 24-, 36-, and 48-month sessions. These longitudinal comparisons were additionally stratified by all starting diagnoses capable of decline after baseline visits (MCI and CN) to further investigate if the ability to predict decline is driven by a specific patient population (Tables 6-7). Similarly to the baseline validation procedures, the results for all comparisons were converted to z-scores, Cohen's d was calculated to assess effect size, and AUC-ROC was computed based on group classification using logistic regression (Tables 6-7).

Longitudinal association with disease severity was tested using linear regression between baseline metric scores and the change in the neuropsychological assessment scores (MMSE, ADAS-11, and CDR-SB) from baseline, at each respective longitudinal session. All absent longitudinal comparisons were excluded due to insufficient sample size.

### Validation Using Clinically Implemented ROI

To validate ADNeuro-Score in the context of the alternative imaging analysis tools frequently used by and accessible to clinicians, the vector of ROIs initially estimated with the FreeSurfer image analysis suite was transformed into a less granular vector of regions that closely resembling the atlas used by Neuroreader®. To investigate how these changes might impact biomarker performance, 80 of the 84 brain regions estimated by FreeSurfer were surjectively mapped to construct the 22 Neuroreader® ROI structures Four of the eighty-four FreeSurfer-estimated regions, the left and right nucleus accumbens and insula, had no corresponding Neuroreader® ROI, and were thus dropped from this branch of the analysis. The previously outlined methods implemented for this analysis were adapted to instead use the pseudo-Neuroreader ROI volumes and were repeated in an otherwise identical manner (Tables 10-13).

### Data Availability

Full, open access to all de-identified ADNI imaging and clinical data is publicly and freely available to individuals who register with the ADNI and agree to the conditions in the “ADNI Data Use Agreement,” upon approval of a request that includes the proposed analysis and the named lead investigator (contact via http://adni.loni.us.edu/data-samples/access-data/). Additional details about the ADNI data acquisition and sharing policies can be found at http://adni.loni.us.edu/wp-content/uploads/how_to_apply/ADNI_DSP_Policy.pdf.

### Results:

### Baseline Validation

### Baseline Sensitivity to Diagnosis

Across all diagnostic group comparisons at baseline, ADNeuro-Score was found to be significantly sensitive to diagnosis (p<0.001 for all comparisons; Table 4). ADNeuro-Score performed best at distinguishing AD from other groups (CN and MCI) and least well at distinguishing CN from MCI participants.

ADNeuro-Score performed as well as the benchmark, AHV (p<0.001 for all comparisons), in this cross-sectional validation (Table 4). AHV also demonstrated a similar performance pattern. AUC-ROC values of ADNeuro-Score and AHV were similar across all group comparisons. However, qualitatively, the overlaid ADNeuro-Score and AHV AUC-ROC curves for AD comparisons (CN vs AD and MCI vs AD) indicated that at low false positive rates, the ADNeuro-Score true positive rate tended to be higher (FIG. 5). The distribution of ADNeuro-Score and AHV for each diagnostic group at baseline are visualized as violin plots (FIG. 6). Notably, ADNeuro-Score qualitatively demonstrated greater separation between diagnostic category medians, with more centrally concentrated distributions. However, at the edges of the distribution, ADNeuro-Score had much longer tails.

Baseline Association with Disease Severity (MMSE, ADAS-11, and CDR-SB)

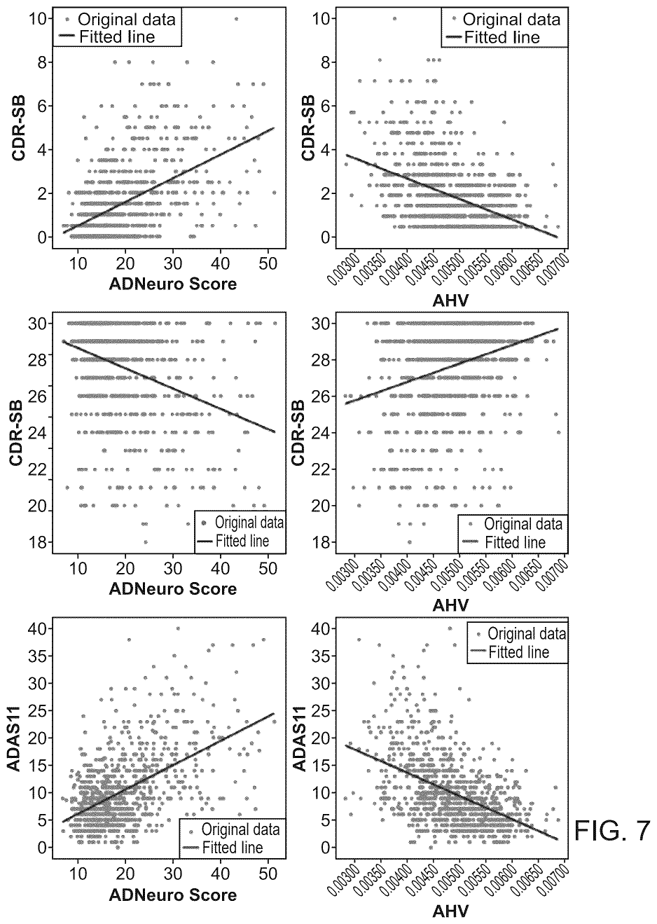

FIG. 7 illustrates overlaid AUC-ROC curves visualizing baseline classification performance of ADNeuro-Score and AHV across the 3 diagnostic group comparisons. In the overall baseline experimental cohort, the ADNeuro-Score was significantly was associated with disease severity, as measured by MMSE, ADAS-11, and CDR-SB scores (Table 5; FIG. 7). Each of these correlations had a slope that was significantly different than zero (p<0.001). A sub-analysis stratified by baseline diagnosis found significant associations between ADNeuro-Score and metrics of disease severity in participants with MCI (MMSE, ADAS-11, and CDR-SB) and AD (ADAS-11, and CDR-SB). Conversely, there was no significant association with disease severity in CN individuals.

ADNeuro-Score generally performed as well as the benchmark, AHV, in this cross-sectional validation using the overall baseline cohort (Table 6; FIG. 7). Results from z-tests conducted using Fisher z-transformed correlation coefficients revealed that correlations between both CDR-SB and ADAS-11 and ADNeuro-Score were significantly stronger than with AHV (p=0.006 for CDR-SB and p=0.024 for ADAS-11). Sub-analyses stratified by baseline diagnosis revealed that ADNeuro-Score and AHV performed the most similarly in participants with MCI, and the most differently in participants with AD (ADNeuro-Score: p<0.001 for ADAS-11 and p<0.01 for CDR-SB, AHV: NS for all).

### Longitudinal Validation

### Metric Sensitivity to Progression

In a longitudinal analysis, the relationship between ADNeuro-Score at baseline and DTC (Diagnosis_{stable}, Diagnosis_{decline}) was assessed at 12-, 24-, 36-, and 48-month sessions. Significant effects of DTC were found at all time points. Stratification by starting diagnosis revealed that ADNeuro-Score's ability to predict decline was primarily driven by individuals with MCI at baseline. Weak to no significant effects were found in CN individuals.

ADNeuro-Score performed equivalently to AHV in the overall experimental cohort. Qualitatively, ADNeuro-Score tended to do modestly better than AHV at differentiating Diagnosis_{stable}, from Diagnosis_{decline} participants at 12- and 24 months, and slightly worse at 36- and 48 months. Sub-analysis by baseline diagnosis revealed similar patterns; ADNeuro-Score performed equivalently to AHV in individuals with MCI. However, AHV demonstrated somewhat higher sensitivity to DTC in the CN group.

Longitudinal Association with Disease Severity (MMSE, ADAS-11, and CDR-SB)

In a longitudinal analysis, the ADNeuro-Score was also significantly associated with the change in disease severity, as measured by the difference in MMSE, ADAS-11, and CDR-SB scores from baseline at each time point (Table 5). Each of these correlations had a slope that was significantly different than zero (p<0.001). Stratification by starting diagnosis revealed that ADNeuro-Score's association with the change in disease severity was also primarily driven by individuals with MCI at baseline. There were significant associations between ADNeuro-Score and the change in MMSE, ADAS-11, and CDR-SB scores in participants with MCI at all time points (p<0.001 for all). Conversely, there was no significant association with the change in disease severity in AD individuals, and only two significant associations in CN individuals (CDR-SB at 12- and 24-months; p<0.001 and p<0.01, respectively).

ADNeuro-Score generally performed as well as the benchmark, AHV, in this longitudinal validation using the overall longitudinal cohort (Tables 5-6). Results from z-tests conducted using Fisher z-transformed correlation coefficients revealed that correlations between ADAS-11 and ADNeuro-Score at month 24 were significantly stronger than with AHV (p=0.003). Sub-analyses stratified by baseline diagnosis revealed that ADNeuro-Score performed as well or somewhat better than AHV in participants with MCI (ADNeuro-Score: p<0.001 for all scores and time points; AHV: p<0.01 for MMSE across all time points and p<0.001 for all other scores and time points). The relationship between MMSE and ADNeuro-Score at month 24 was significantly stronger than with AHV (p=0.040).

The CN sub-analysis and AD sub-analysis were limited by sample size longitudinally. The correlations between both metrics and the neuropsychological scale scores in participants with AD were similarly weak and insignificant across all sessions, with the exception of the AHV-ADAS-11 correlation at month 12 (p<0.01). In the CN group, ADNeuro-Score and AHV again performed similarly, with generally weak correlations that were all insignificant aside from ADNeuro-Score and CDR-SB at 12-(p<0.001) and 24-months (p<0.01), and AHV and MMSE at 48-months (p<0.05), which was significantly stronger than with ADNeuro-Score (p=0.044).

### Alternative Atlas Analysis

### Baseline and Longitudinal Validation

All analyses repeated using the alternative Neuroreader® atlas ROIs produced similar trends to those obtained with FreeSurfer, both at baseline and across longitudinal sessions. Performance of ADNeuro-score and AHV were analogous to those of the original analysis, the results of which are summarized in Tables 11-13.

### Discussion (Target 920 or Less)

Currently, the gold standard for diagnosing AD and quantifying disease severity is neurocognitive examination. The safety, convenience, and widespread availability of sMRI paired with its ability to quantify neurodegeneration have made this tool valuable in both clinical and research setting. Recent decades have seen a surge in the amount, sophistication, and precision of automated methods used to extract brain volumes and perform subcortical segmentation (Bishop et al., 2011; Bozzali et al., 2006; Feczko et al., 2009; Ferreira et al., 2011; Firbank et al., 2008; Karas et al., 2003; Kehoe et al., 2014; Whitwell et al., 2008). sMRI-estimated atrophy offers an independent measure of AD pathology that can provide important, additional information over neurocognitive assessment alone. Volumetric metrics are a sensitive to neuronal damage, while the scores neuropsychological scales may not detect all AD pathology due to factors like cognitive reserve. Further, volumetric measures are not subject to floor or ceiling effects. Additionally, in patients in the later, more severe stages of AD, there may be less clinical value in performing full neurocognitive assessments, sMRI-based metrics could be useful way to track disease advancement with lower patient and clinical burden.

A cross-sectional validation revealed that ADNeuro-Score was significantly associated with diagnosis and disease severity. Moreover, ADNeuro-Score again performed as well as our benchmark, AHV, and demonstrated comparably strong correlations with MMSE, ADAS-11 and CDR-SB scores, which were statistically significantly stronger for the latter two assessments. Longitudinally, trends in correlation coefficient magnitude and significance between ADNeuro-Score and the change in the 3 neuropsychological scale scores persisted across all time points; however, stratifying the longitudinal metric correlations by initial diagnostic group revealed that the MCI patient population was largely driving the correlations found in the overall longitudinal experimental cohort. In the MCI sub-group, the ADNeuro-Score demonstrated similar or significantly stronger longitudinal correlations with ADAS-11, MMSE, and CDR-SB scores, compared to AHV. This is primarily a consequence of the limited sample size of CN participants and patients with AD with longitudinal scores available, which rendered the CN and AD sub-analyses underpowered to detect a relationship. Second, some degree of randomness or noise can be said to be introduced by the patients with a baseline CN diagnosis, because this diagnostic class is comprised of two distinct sub-populations: a number of patients early along the trajectory of AD which present detectable differences in brain morphology that have not yet manifested as clinically observable symptoms, and participants that are representative of the typical, healthy average brain.

Present approaches further relate to a Euclidean-inspired sMRI-based distance metric, AD-NeuroScore, which was significantly associated with diagnosis (CN, MCI, and AD) and disease severity (MMSE, ADAS-11 and CDR-SB scores) at baseline. AD-NeuroScore performed as well as AHV, the most commonly used sMRI measure of AD-related neurodegeneration, and demonstrated comparably strong correlations with disease severity scores. AD-NeuroScore was also significantly associated with changes in the three disease severity scores over a relatively long follow-up period of 48 months. These associations are largely driven by the MCI group. It is possible that the limited sample size of CN participants and patients with AD with longitudinal scores available resulted in the CN and AD sub-analyses being underpowered to detect a relationship.

It is also important to address the distinctions between AD-NeuroScore and other potentially similarly inspired biomarkers. The STructural Abnormality iNDex (STAND)-score is a result of a support vector machine classifier which takes in sMRI normalized input features such as GM, WM, and CSF tissue densities extracted from SPM5 tissue segmentation as well as demographic information, and assigns a numerical value used to classify a patient as CN or AD based on a 280 sample (140 each) training set from the Alzheimer's disease Patient Registry (Vemuri et al., 2009b, n.d.). Another similar metric, AD Pattern Similarity (AD-PS), uses the same sMRI input features (GM, WM, and CSF tissue maps) derived from the ADNI data set (Casanova et al., 2013). AD-PS uses high dimensional regularized logistic regression to classify a patient as CN or AD. AD-PS and AD-NeuroScore perform similarly (with significantly overlapping AUC-ROC confidence intervals); while AD-PS was also shown to correlate negatively with cognitive scores obtained through clinic visits and telephone interviews, lack of details regarding the correlations makes direct comparison with AD-NeuroScore difficult.

Thus, many of the ML approaches, including STAND and AD-PS, have demonstrated their potential to successfully classify participants as AD or not, using similar input features (perhaps from different data sets) and slightly different ML algorithms; however, they possess nearly identical deficiencies, primarily concerning their difficult interpretability. Other modifications to this black box technique utilize limited or full sets brain volumes such as hippocampal grading or MTL scoring methods as opposed to voxel-based morphometry, all of which, again, present similar bottlenecks for clinical utility as the other black box methods (Arimura et al., 2008; Coupé et al., 2015e). One unique approach, MRDATS, uses w-corrected brain volumes in an ensemble-learning algorithm to give a score between 0 and 1 to indicate CN to Dementia progression (Popuri et al., 2020b). Using a threshold of 0.5, MRDATS performed similarly to the aforementioned methods at binary classification; however, this metric is bounded, making it less optimally suited to describe and differentiate individuals at more advanced disease stages.

Longitudinal analyses of the distribution of ADNeuro-Score between groups that were stable or experienced a decline in diagnosis from baseline found significant effects of DTC at all time points that was equivalent to the performance of AHV. Qualitatively, ADNeuro-Score tended to perform modestly better than AHV at earlier time points (12- and 24-months) and slightly worse at later sessions (36- and 48-months). The finding may be partly attributable to the much larger sample size of patients at the earlier time points, which conferred greater statistical power to detect association with progression. Stratifying this analysis by starting diagnosis revealed that the MCI patient population was primarily driving these results as well. This finding is again likely the result of two major factors; the combined sample of stable and declining patients at each time point was always much larger for the patients with a baseline MCI diagnosis and the greatest and most consistent changes in neuropsychological scale scores across sessions would be expected to occur in the MCI sub-population. Because patients more frequently seek monitoring and intervention after the onset of cognitive deficits (i.e. with MCI) rather than prior (i.e. CN), the strength of our metric in this patient population is of importance. Similarly, in clinical trials aimed at assessing the efficacy of interventions in slowing AD progression, a measure that is optimally sensitive in patients with MCI is particularly well-suited for usage.

An unexpected result of the present study, inconsistent with previous literature, is that both the left and right lateral ventricular volumes were not retained during ROI selection, despite the frequent observation of ventricular dilation in patients with AD (Attier-Zmudka et al., 2019; Ott et al., 2010), which often occurs as a result of reduced cerebrospinal fluid turnover, likely caused by impaired amyloid beta peptide clearance in AD-related neuropathology (Nestor et al., 2008a). Further, absolute ventricular enlargement has proven sufficient to discriminate between MCI patients who progressed to AD or remained stable over a 6-month interval in a smaller ADNI cohort (n=504) and, consequently, has been proposed as a potential biomarker for disease progression (Nestor et al., 2008b). However, at baseline, in line with previous cross-sectional volumetric studies, a large overlap was found between the total ventricular volumes of pathological groups and controls (Giesel et al., n.d.; Nestor et al., 2008b; Schott et al., 2005). Thus, one potential explanation may be that absolute ventricular change, more frequently employed as a primary outcome measure in studies focused on understanding this relationship, is a more sensitive measure of AD-pathology than total ventricular volume. Given that our investigation only examined regional volumes at cross-section and employed a conservative correction to adjust for the 84 brain regions tested during ROI selection, this discrepancy may be plausibly attributed to differences in study design. A particularly attractive feature of ADNeuro-Score is the ease with which it could be integrated into current clinical and research workflows. Obtaining ADNeuro-Score would be a simple addition to existing, clinically-implemented regional brain volume reports, and similarly, in research, would be as simple to acquire as the individual regional volumes commonly used as endpoints. Further, by replacing different anatomical ROIs with a single measure as an endpoint, ADNeuro-Score could enhance the sensitivity of many studies by reducing the number of statistical tests, while meaningfully encapsulating as much or more information. Future directions of this work include creating validated ADNeuro-Score norms and diagnostic cutoffs to aid in integration with clinical workflows. Additionally, ADNeuro-Score may be utilized as an endpoint in clinical research. If sufficiently sensitive enough to detect effects, ADNeuro-Score could be useful in increasing study-wide statistical power by reducing the number of sMRI-based study endpoints and corresponding number of comparisons.

An advantage of ADNeuro-Score is its potential to accurately monitor disease advancement and be compared across multiple sites by using known ranges to generate probabilities of diagnosis or decline. Similarly, expected ranges of ADNeuro-Score that are standard for different groups could be used to aid early differential diagnosis of common neurodegenerative diseases that cause dementia, such as Vascular dementia (VaD), dementia with Lewy bodies (DLB), and frontotemporal dementia (FTD), which demonstrate unique patterns of atrophy that AHV and other standard approaches are unequipped to assess. In support of exploring this functionality further is previous literature which has found that sMRI alone can be used to differentiate between these four dementia-causing neurodegenerative diseases groups and healthy controls with a high accuracy (Koikkalainen et al., 2016). A future direction of this research is to determine if ADNeuro-Score can aid in the differential diagnosis of dementia.

While the findings of this work encourage further investigation, there are several limitations that should be carefully considered and addressed in successive studies. First, the sample of patients with AD was much smaller than the CN and MCI cohorts across all sessions, with this number falling across successive visits. The limited number of participants across all diagnostic groups with scores available on the MMSE, CDR-SB, and ADAS-11 also dwindled longitudinally, rendering some of the later tests for the CN and AD group particularly underpowered. Additionally, the AD subset was confined to patients with mild AD dementia, as defined in the ADNI protocol, which leaves more progressed patients with AD understudied with regards to the biomarker. Repeating this study in a real-world clinical sample that better represents the AD patient class at various stages of disease advancement is another future direction of this work.

Table 3. Significant ROIs by Z-Score Ranking and CN Template Values: Resulting 41 significant regions of interest (ROIs) extracted by performing ANOVA for each of the 84 regions in the ROI selection cohort are reported in the above table along with corresponding z scores. Significance was established based on an alpha=0.05, Bonferroni corrected. Structures are identified by both the FreeSurfer version 7.1 ROI labels and conventional anatomical names. Mean volumes and standard deviations (SD) of the CN template cohort are included for each respective region.

| | | | CN Template Mean |
|---|---|---|---|
| FreeSurfer ROI Label | Anatomical Name | Z | Vol (±SD) (mm³) |
| Lhippo | Left Hippocampus | 9.12 | 0.48 (±0.74) |
| Lamyg | Left Amygdala | 8.43 | 0.60 (±0.79) |
| Ramyg | Right Amygdala | 8.37 | 0.54 (±0.80) |
| Rhippo | Right Hippocampus | 7.82 | 0.45 (±0.71) |
| L_middletemporal_grayvol | Left Middle Temporal Lobe | 6.53 | 0.40 (±0.88) |
| L_fusiform_grayvol | Left Fusiform Gyrus | 6.20 | 0.27 (±0.82) |
| L_inferiorparietal_grayvol | Left Inferior Parietal Lobe | 6.11 | 0.33 (±0.90) |
| R_middletemporal_grayvol | Right Middle Temporal Lobe | 6.12 | 0.42 (±0.92) |
| L_inferiortemporal_grayvol | Left Inferior Temporal Lobe | 5.88 | 0.36 (±0.85) |
| L_precuneus_grayvol | Left Precuneus | 5.81 | 0.26 (±0.91) |
| L_rostralmiddlefrontal_grayvol | Left Rostral Middle Frontal Lobe | 5.69 | 0.16 (±1.06) |
| R_inferiortemporal_grayvol | Right Inferior Temporal Lobe | 5.54 | 0.27 (±0.87) |
| R_precuneus_grayvol | Right Precuneus | 5.50 | 0.34 (±0.93) |
| R_inferiorparietal_grayvol | Right Inferior Parietal Lobe | 5.48 | 0.33 (±0.88) |
| R_superiortemporal_grayvol | Right Superior Temporal Lobe | 5.44 | 0.35 (±0.94) |
| R_entorhinal_grayvol | Right Entorhinal Cortex | 5.39 | 0.12 (±0.77) |
| Lthal | Left Thalamus | 5.1 | 0.33 (±1.01) |
| R_lateralorbitofrontal_grayvol | Right Lateral Orbitofrontal Cortex | 5.02 | 0.14 (±0.91) |
| L_superiortemporal_grayvol | Left Superior Temporal Lobe | 5.01 | 0.28 (±0.87) |
| L_insula_grayvol | Left Insula | 4.86 | 0.19 (±0.86) |
| L_bankssts_grayvol | Left Banks of the Superior Temporal Sulcus | 4.75 | 0.12 (±0.93) |
| L_lateralorbitofrontal_grayvol | Left Lateral Orbitofrontal Cortex | 4.67 | 0.26 (±0.89) |
| L_isthmuscingulate_grayvol | Left Isthmus of the Cingulate Gyrus | 4.66 | 0.20 (±1.04) |
| R_parahippocampal_grayvol | Right Parahippocampal Gyrus | 4.64 | 0.21 (±0.91) |
| Raccumb | Right Nucleus Accumbens | 4.58 | 0.33 (±0.88) |
| L_entorhinal_grayvol | Left Entorhinal Cortex | 4.56 | 0.23 (±0.74) |
| R_rostralmiddlefrontal_grayvol | Right Rostral Middle Frontal Gyrus | 4.50 | 0.18 (±1.04) |
| L_superiorparietal_grayvol | Left Superior Parietal Lobe | 4.44 | 0.14 (±0.87) |
| Rthal | Right Thalamus | 4.42 | 0.31 (±1.02) |
| L_superiorfrontal_grayvol | Left Superior Frontal Gyrus | 4.38 | 0.17 (±0.89) |
| R_fusiform_grayvol | Right Fusiform Gyrus | 4.25 | 0.23 (±0.88) |
| R_insula_grayvol | Right Insula | 4.14 | 0.21 (±0.93) |
| L_lateraloccipital_grayvol | Left Lateral Occipital Cortex | 4.08 | 0.31 (±0.87) |
| R_superiorfrontal_grayvol | Right Superior Frontal Gyrus | 3.96 | 0.16 (±0.99) |
| L_posteriorcingulate grayvol | Left Posterior Cingulate Cortex | 3.86 | 0.29 (±0.98) |
| R_isthmuscingulate_grayvol | Right Isthmus of the Cingulate Gyrus | 3.78 | 0.13 (±0.87) |
| R_superiorparietal_grayvol | Right Superior Parietal Lobe | 3.75 | 0.26 (±1.00) |
| R_parsorbitalis_grayvol | Pars Orbitalis | 3.70 | 0.19 (±0.92) |
| R_posteriorcingulate_grayvol | Right Posterior Cingulate Cortex | 3.60 | 0.24 (±0.90) |
| Laccumb | Left Nucleus Accumbens | 3.60 | 0.45 (±1.04) |
| R_lateraloccipital_grayvol | Right Lateral Occipital Cortex | 3.47 | 0.24 (±0.83) |

Table 4. Baseline Results for ADNeuro-Score Sensitivity to Diagnosis: Sensitivity to diagnosis assessed using pairwise, two-tailed t-tests performed for each possible diagnostic group comparison. Resulting z-scores, effect sizes (Cohen's d) with 95% confidence intervals (CI) s, and AUC-ROC values with 95% CI performance are included. Results using AHV are included for benchmarking. Significant results from group comparisons are denoted by * to indicate p<0.05, ** to indicate p<0.01, and *** to indicate p<0.001, Holm-Bonferroni corrected.

| Group Comparison | Metric | AUC [95% CI] | Cohen's d [95% CI] | z-score |
|---|---|---|---|---|
| CN vs MCI | AD-NeuroScore | 0.66 [0.59, 0.73] | −0.56 [−0.59, −0.53]*** | −7.46 |
| | AHV | 0.65 [0.58, 0.72] | 0.55 [0.52, 0.58]*** | −7.27 |
| CN vs AD | AD-NeuroScore | 0.91 [0.86, 0.96] | −2.06 [−2.24, −1.88]*** | −16.37 |
| | AHV | 0.88 [0.81, 0.95] | 1.72 [1.55, 1.89]*** | −14.27 |
| MCI vs AD | AD-NeuroScore | 0.80 [0.71, 0.89] | −1.16 [−1.19, −1.14]*** | −11.23 |
| | AHV | 0.76 [0.67, 0.84] | 0.92 [0.90, 0.94]*** | −9.04 |

Table 5. Relationship Between AD-NeuroScore and Disease Severity. Baseline rows include results from cross-sectional analysis of AD-NeuroScore and disease severity, operationalized as MMSE, ADAS-11, and CDR-SB scores. All other rows include results from longitudinal analysis of baseline AD-NeuroScore and change in disease severity scores at 12, 24, 36, and 48 months. Testing was performed with linear regression in the overall experimental cohort and in each diagnostic sub-group. Performance of AHV is included for benchmarking. Significant associations are indicated by * for p<0.05, ** for p<0.01, and *** for p<0.001, Holm-Bonferroni corrected.

| | | | r | | | n | | |
|---|---|---|---|---|---|---|---|---|
| Class | t | Metric | CDR-SB | MMSE | ADAS-11 | CDR-SB | MMSE | ADAS-11 |
| All | Baseline | ADNeuro-Score | 0.52 | −0.34*** | 0.53*** | 926 | 926 | 926 |
| | | AHV | −0.43*** | 0.28*** | −0.47*** | 926 | 926 | 926 |
| | 12 | ADNeuro-Score | −0.27*** | 0.21*** | −0.23*** | 525 | 697 | 721 |
| | | AHV | 0.23*** | −0.16*** | 0.19*** | 525 | 697 | 721 |
| | 24 | ADNeuro-Score | −0.40*** | 0.27*** | −0.39*** | 402 | 614 | 626 |
| | | AHV | 0.32*** | −0.19*** | 0.26*** | 402 | 614 | 626 |
| | 36 | ADNeuro-Score | −0.38*** | 0.21*** | −0.31*** | 285 | 332 | 345 |
| | | AHV | 0.34*** | −0.17** | 0.26*** | 285 | 332 | 345 |
| | 48 | ADNeuro-Score | −0.42*** | 0.26*** | −0.31*** | 205 | 369 | 375 |
| | | AHV | 0.35*** | −0.19*** | 0.26*** | 205 | 369 | 375 |
| CN | Baseline | ADNeuro-Score | −0.08 | −0.01 | 0.10 | 286 | 286 | 286 |
| | | AHV | 0.00 | −0.01 | −0.17** | 286 | 286 | 286 |
| | 12 | ADNeuro-Score | −0.81*** | −0.02 | 0.09 | 13 | 183 | 180 |
| | | AHV | 0.53 | −0.03 | 0.01 | 13 | 183 | 180 |
| | 24 | ADNeuro-Score | −0.69** | 0.02 | −0.09 | 17 | 220 | 221 |
| | | AHV | 0.45 | −0.08 | 0.05 | 17 | 220 | 221 |
| | 36 | ADNeuro-Score | — | −0.06 | 0.01 | 1 | 44 | 36 |
| | | AHV | — | −0.19 | 0.03 | 1 | 44 | 36 |
| | 48 | ADNeuro-Score | −0.12 | 0.00 | 0.01 | 5 | 126 | 133 |
| | | AHV | 0.52 | −0.21* | −0.01 | 5 | 126 | 133 |
| MCI | Baseline | ADNeuro-Score | 0.23*** | −0.09* | 0.33*** | 511 | 511 | 511 |
| | | AHV | −0.22*** | 0.11* | −0.35*** | 511 | 511 | 511 |
| | 12 | ADNeuro-Score | −0.30*** | 0.21*** | −0.19*** | 418 | 423 | 452 |
| | | AHV | 0.23*** | −0.13** | 0.17*** | 418 | 423 | 452 |
| | 24 | ADNeuro-Score | −0.39*** | 0.28*** | −0.34*** | 357 | 357 | 377 |
| | | AHV | 0.32*** | −0.15** | 0.25*** | 357 | 357 | 377 |
| | 36 | ADNeuro-Score | −0.38*** | 0.24*** | −0.31*** | 284 | 273 | 309 |
| | | AHV | 0.35*** | −0.16** | 0.27*** | 284 | 273 | 309 |
| | 48 | ADNeuro-Score | −0.42** | 0.30*** | −0.31*** | 199 | 226 | 242 |
| | | AHV | 0.35*** | −0.19** | 0.28*** | 199 | 226 | 242 |
| AD | Baseline | ADNeuro-Score | 0.27** | −0.17 | 0.33*** | 129 | 129 | 129 |
| | | AHV | −0.08 | 0.08 | −0.14 | 129 | 129 | 129 |
| | 12 | ADNeuro-Score | 0.09 | 0.04 | 0.00 | 94 | 91 | 89 |
| | | AHV | −0.08 | −0.03 | −0.22* | 94 | 91 | 89 |
| | 24 | ADNeuro-Score | 0.03 | 0.16 | −0.36 | 28 | 37 | 28 |
| | | AHV | −0.34 | −0.10 | −0.20 | 28 | 37 | 28 |
| | 36 | ADNeuro-Score | — | −0.13 | — | 0 | 15 | 0 |
| | | AHV | — | −0.09 | — | 0 | 15 | 0 |
| | 48 | ADNeuro-Score | — | 0.28 | — | 1 | 17 | 0 |
| | | AHV | — | −0.11 | — | 1 | 17 | 0 |

Table 6. Relationship Between ADNeuro-Score and Longitudinal Diagnosis Transitions: Sensitivity to diagnostic transition assessed using pairwise, two-tailed t-tests performed for each possible diagnostic group comparison and for all follow-up time points. Resulting z-scores, effect sizes (Cohen's d) with 95% confidence intervals (CI) s, and AUC-ROC values with 95% CI performance are included. Performance was evaluated both in the overall experimental cohort and in sub-groups based on starting diagnosis (CN or MCI). Results using AHV are included for benchmarking. Significant results from group comparisons are denoted by * to indicate p<0.05, ** to indicate p<0.01, and *** to indicate p<0.001. Significant group comparisons are denoted by * to indicate p<0.05, ** to indicate p<0.01, and *** to indicate p<0.001, Holm-Bonferroni corrected.

| Comparison | | t | Metric | AUC [95% CI] | Cohen's d [95% CI] | | z-score |
|---|---|---|---|---|---|---|---|
| Stable | All | 12 months | ADNS | 0.67 [0.53, 0.80] | 0.64 | [0.35, 0.93]*** | −4.35 |
| vs | | | AHV | 0.65 [0.50, 0.80] | −0.53 | [−0.81, −0.24]*** | −3.57 |
| Decline | | 24 months | ADNS | 0.71 [0.59, 0.82] | 0.84 | [0.61, 1.08]*** | −7.00 |
| | | | AHV | 0.71 [0.61, 0.82] | −0.79 | [−1.02, −0.55]*** | −6.58 |
| | | 36 months | ADNS | 0.69 [0.53, 0.85] | 0.72 | [0.46, 0.98]*** | −5.42 |
| | | | AHV | 0.72 [0.60, 0.85] | −0.79 | [−1.05, −0.52]*** | −5.88 |
| | | 48 months | ADNS | 0.67 [0.52, 0.83] | 0.74 | [0.47, 1.01]*** | −5.36 |
| | | | AHV | 0.72 [0.62, 0.83] | −0.79 | [−1.06, −0.51]*** | −5.66 |
| | CN | 12 months | ADNS | 0.35 [0.08, 0.62] | −0.06 | [−0.70, 0.59] | −0.17 |
| | | | AHV | 0.72 [0.35, 1.09] | −0.59 | [−1.23, 0.06] | −1.79 |
| | | 24 months | ADNS | 0.64 [0.41, 0.88] | 0.55 | [0.08, 1.01]* | −2.32 |
| | | | AHV | 0.74 [0.53, 0.95] | −0.79 | [−1.26, −0.33]*** | −3.34 |
| | | 36 months | ADNS | 0.59 [0.21, 0.97] | 0.34 | [−0.36, 1.05] | −0.98 |
| | | | AHV | 0.78 [0.46, 1.09] | −0.84 | [−1.57, −0.11]* | −2.30 |
| | | 48 months | ADNS | 0.44 [0.14, 0.75] | 0.11 | [−0.44, 0.65] | −0.39 |
| | | | AHV | 0.72 [0.46, 0.98] | −0.65 | [−1.20, −0.10]* | −2.34 |
| | MCI | 12 months | ADNS | 0.77 [0.65, 0.90] | 1.08 | [0.75, 1.42]*** | −6.39 |
| | | | AHV | 0.68 [0.51, 0.85] | −0.69 | [−1.02, −0.36]*** | −4.14 |
| | | 24 months | ADNS | 0.76 [0.64, 0.88] | 1.05 | [0.77, 1.34]*** | −7.36 |
| | | | AHV | 0.73 [0.59, 0.86] | −0.85 | [−1.12, −0.57]*** | −5.99 |
| | | 36 months | ADNS | 0.73 [0.62, 0.84] | 0.87 | [0.59, 1.16]*** | −5.99 |
| | | | AHV | 0.73 [0.56, 0.90] | −0.86 | [−1.14, −0.57]*** | −5.87 |
| | | 48 months | ADNS | 0.73 [0.57, 0.89] | 0.78 | [0.46, 1.10]*** | −4.83 |
| | | | AHV | 0.69 [0.51, 0.88] | −0.76 | [−1.08, −0.44]*** | −4.71 |

Table 7. Baseline Assessment of all Distance Metrics based on Sensitivity to Diagnosis. Assessment of all distance metrics, benchmarked against AHV, based on metric sensitivity to diagnosis at baseline, operationalized as pairwise, two-tailed t-tests performed for each possible diagnostic group comparison, z-scores, effect sizes (Cohen's d) with 95% CIs, and AUC values with 95% CIs. AD-NeuroScore (ADNS) is used to refer to the Z-Weighted Euclidean (ZWE) distance function. The Fréchet and Hausdorff distance functions were found to be the same in one dimension. Significant group comparisons are denoted by * to indicate p<0.05, ** to indicate p<0.01, and *** to indicate p<0.001.

| Group Comparison | Metric | AUC [95% CI] | Cohen's d [95% CI] | z-score |
|---|---|---|---|---|
| CN vs MCI | ADNS | 0.66 [0.59, 0.73] | −0.56 [−0.59, −0.53]*** | −7.46 |
| | Euclidean | 0.64 [0.55, 0.73] | −0.47 [−0.50, −0.44]*** | −6.31 |
| | 1-D Fréchet | 0.65 [0.57, 0.73] | −0.49 [−0.52, −0.46]*** | −6.57 |
| | N-D Fréchet | 0.58 [0.49, 0.66] | −0.33 [−0.35, −0.30]*** | −4.41 |
| | 1-D Hausdorff | 0.63 [0.55, 0.71] | −0.49 [−0.52, −0.46]*** | −6.57 |
| | N-D Hausdorff | 0.56 [0.49, 0.63] | −0.22 [−0.24, −0.19]** | −2.95 |
| | AHV | 0.65 [0.58, 0.72] | 0.55 [0.52, 0.58]*** | −7.27 |
| CN vs AD | ADNS | 0.91 [0.86, 0.96] | −2.06 [−2.24, −1.88]*** | −16.37 |
| | Euclidean | 0.85 [0.78, 0.92] | −1.44 [−1.60, −1.28]*** | −12.32 |
| | 1-D Fréchet | 0.85 [0.78, 0.93] | −1.57 [−1.73, −1.40]*** | −13.23 |
| | N-D Fréchet | 0.80 [0.71, 0.88] | −1.28 [−1.44, −1.12]*** | −11.18 |
| | 1-D Hausdorff | 0.86 [0.79, 0.93] | −1.57 [−1.73, −1.40]*** | −13.23 |
| | N-D Hausdorff | 0.73 [0.62, 0.84] | −0.93 [−1.08, −0.78]*** | −8.40 |
| | AHV | 0.88 [0.81, 0.95] | 1.72 [1.55, 1.89]*** | −14.27 |
| MCI vs AD | ADNS | 0.80 [0.71, 0.89] | −1.16 [−1.19, −1.14]*** | −11.23 |
| | Euclidean | 0.74 [0.64, 0.85] | −1.02 [−1.05, −1.00]*** | −9.98 |
| | 1-D Fréchet | 0.74 [0.64, 0.84] | −1.02 [−1.05, −1.00]*** | −9.99 |
| | N-D Fréchet | 0.73 [0.63, 0.83] | −0.85 [−0.87, −0.83]*** | −8.41 |
| | 1-D Hausdorff | 0.74 [0.63, 0.84] | −1.02 [−1.05, −1.00]*** | −9.99 |
| | N-D Hausdorff | 0.64 [0.53, 0.75] | −0.68 [−0.71, −0.66]*** | −6.82 |
| | AHV | 0.76 [0.67, 0.84] | 0.92 [0.90, 0.94]*** | −9.04 |

Table 8. Linear Regression Results All Distance Metrics. Assessment of all distance metrics, benchmarked against AHV, on the basis of metric association with disease severity, operationalized as MMSE, ADAS-11, and CDR-SB scores at baseline or their change from baseline at each time point, tested with linear regression in the overall experimental cohort and in each diagnostic sub-group. ADNS is used here to refer to the Z-Weighted Euclidean (ZWE) distance function. The Fréchet and Hausdorff distance functions were found to be the same in one dimension. Significant group comparisons are denoted by * to indicate p<0.05, ** to indicate p<0.01, and *** to indicate p<0.001.

| | | | r | | | n | | |
|---|---|---|---|---|---|---|---|---|
| Class | t | Metric | CDR-SB | MMSE | ADAS-11 | CDR-SB | MMSE | ADAS-11 |
| All | Baseline | ADNS | 0.52*** | −0.34*** | 0.53*** | 926 | 926 | 926 |
| | | Euclidean | 0.47*** | −0.32*** | 0.49*** | 926 | 926 | 926 |
| | | 1-D Fréchet | 0.47*** | −0.31*** | 0.49*** | 926 | 926 | 926 |
| | | N-D Fréchet | 0.39*** | −0.26*** | 0.42*** | 926 | 926 | 926 |
| | | 1-D Hausdorff | 0.47*** | −0.31*** | 0.49*** | 926 | 926 | 926 |
| | | N-D Hausdorff | 0.33*** | −0.22*** | 0.33*** | 926 | 926 | 926 |
| | | AHV | −0.43*** | 0.28*** | −0.47*** | 926 | 926 | 926 |
| | 12 | ADNS | −0.27*** | 0.21*** | −0.23*** | 525 | 697 | 721 |
| | | Euclidean | −0.23*** | 0.22*** | −0.20*** | 525 | 697 | 721 |
| | | 1-D Fréchet | −0.24*** | 0.22*** | −0.20*** | 525 | 697 | 721 |
| | | N-D Fréchet | −0.21*** | 0.16*** | −0.19*** | 525 | 697 | 721 |
| | | 1-D Hausdorff | −0.24*** | 0.22*** | −0.20*** | 525 | 697 | 721 |
| | | N-D Hausdorff | −0.15*** | 0.08* | −0.12** | 525 | 697 | 721 |
| | | AHV | 0.23*** | −0.16*** | 0.19*** | 525 | 697 | 721 |
| | 24 | ADNS | −0.40*** | 0.27*** | −0.39*** | 402 | 614 | 626 |
| | | Euclidean | −0.34*** | 0.24*** | −0.39*** | 402 | 614 | 626 |
| | | 1-D Fréchet | −0.34*** | 0.24*** | −0.36*** | 402 | 614 | 626 |
| | | N-D Fréchet | −0.29*** | 0.19*** | −0.29*** | 402 | 614 | 626 |
| | | 1-D Hausdorff | −0.34*** | 0.24*** | −0.36*** | 402 | 614 | 626 |
| | | N-D Hausdorff | −0.22*** | 0.13** | −0.23*** | 402 | 614 | 626 |
| | | AHV | 0.32*** | −0.19*** | 0.26*** | 402 | 614 | 626 |
| | 36 | ADNS | −0.38*** | 0.21*** | −0.31*** | 285 | 332 | 345 |
| | | Euclidean | −0.33*** | 0.18** | −0.25*** | 285 | 332 | 345 |
| | | 1-D Fréchet | −0.33*** | 0.18** | −0.26*** | 285 | 332 | 345 |
| | | N-D Fréchet | −0.25*** | 0.11* | −0.17** | 285 | 332 | 345 |
| | | 1-D Hausdorff | −0.33*** | 0.18** | −0.26*** | 285 | 332 | 345 |
| | | N-D Hausdorff | −0.19** | 0.08 | −0.10 | 285 | 332 | 345 |
| | | AHV | 0.34*** | −0.17** | 0.26*** | 285 | 332 | 345 |
| | 48 | ADNS | −0.42*** | 0.26*** | −0.31*** | 205 | 369 | 375 |
| | | Euclidean | −0.39*** | 0.20*** | −0.25*** | 205 | 369 | 375 |
| | | 1-D Fréchet | −0.37*** | 0.20*** | −0.24*** | 205 | 369 | 375 |
| | | N-D Fréchet | −0.33*** | 0.15** | −0.18*** | 205 | 369 | 375 |
| | | 1-D Hausdorff | −0.37*** | 0.20*** | −0.24*** | 205 | 369 | 375 |
| | | N-D Hausdorff | −0.27*** | 0.10 | −0.13* | 205 | 369 | 375 |
| | | AHV | 0.35*** | −0.19*** | 0.26*** | 205 | 369 | 375 |
| CN | Baseline | ADNS | −0.08 | −0.01 | 0.10 | 286 | 286 | 286 |
| | | Euclidean | −0.08 | 0.00 | 0.11 | 286 | 286 | 286 |
| | | 1-D Fréchet | −0.09 | 0.01 | 0.10 | 286 | 286 | 286 |
| | | N-D Fréchet | −0.03 | −0.05 | 0.11 | 286 | 286 | 286 |
| | | 1-D Hausdorff | −0.09 | 0.01 | 0.1 | 286 | 286 | 286 |
| | | N-D Hausdorff | 0.01 | −0.11 | 0.11 | 286 | 286 | 286 |
| | | AHV | 0.00 | −0.01 | −0.17** | 286 | 286 | 286 |
| | 12 | ADNS | −0.81*** | −0.02 | 0.09 | 13 | 183 | 180 |
| | | Euclidean | −0.86*** | −0.01 | 0.05 | 13 | 183 | 180 |
| | | 1-D Fréchet | −0.82*** | 0.00 | 0.04 | 13 | 183 | 180 |
| | | N-D Fréchet | −0.60* | 0.00 | 0.01 | 13 | 183 | 180 |
| | | 1-D Hausdorff | −0.82*** | 0.00 | 0.04 | 13 | 183 | 180 |
| | | N-D Hausdorff | −0.53 | 0.02 | −0.05 | 13 | 183 | 180 |
| | | AHV | 0.53 | −0.03 | 0.01 | 13 | 183 | 180 |
| | 24 | ADNS | −0.69** | 0.02 | −0.09 | 17 | 220 | 221 |
| | | Euclidean | −0.79*** | 0.12 | −0.06 | 17 | 220 | 221 |
| | | 1-D Fréchet | −0.74*** | 0.14* | −0.07 | 17 | 220 | 221 |
| | | N-D Fréchet | −0.62** | 0.14* | −0.11 | 17 | 220 | 221 |
| | | 1-D Hausdorff | −0.74*** | 0.14* | −0.07 | 17 | 220 | 221 |
| | | N-D Hausdorff | −0.49* | 0.10 | −0.11 | 17 | 220 | 221 |
| | | AHV | 0.45 | −0.08 | 0.05 | 17 | 220 | 221 |
| | 36 | ADNS | — | −0.06 | 0.01 | 1 | 44 | 36 |
| | | Euclidean | — | −0.09 | −0.06 | 1 | 44 | 36 |
| | | 1-D Fréchet | — | −0.08 | −0.08 | 1 | 44 | 36 |
| | | N-D Fréchet | — | −0.08 | −0.17 | 1 | 44 | 36 |
| | | 1-D Hausdorff | — | −0.08 | −0.08 | 1 | 44 | 36 |
| | | N-D Hausdorff | — | 0.12 | 0.05 | 1 | 44 | 36 |
| | | AHV | — | −0.19 | 0.03 | 1 | 44 | 36 |
| | 48 | ADNS | −0.12 | 0.00 | 0.01 | 5 | 126 | 133 |
| | | Euclidean | 0.03 | 0.00 | 0.07 | 5 | 126 | 133 |
| | | 1-D Fréchet | 0.02 | 0.02 | 0.06 | 5 | 126 | 133 |
| | | N-D Fréchet | −0.28 | 0.03 | 0.02 | 5 | 126 | 133 |
| | | 1-D Hausdorff | 0.02 | 0.02 | 0.06 | 5 | 126 | 133 |
| | | N-D Hausdorff | 0.29 | 0.04 | −0.13 | 5 | 126 | 133 |
| | | AHV | 0.52 | −0.21* | −0.01 | 5 | 126 | 133 |
| MCI | Baseline | ADNS | 0.23*** | −0.09* | 0.33*** | 511 | 511 | 511 |
| | | Euclidean 1-D | 0.20*** | −0.08 | 0.28*** | 511 | 511 | 511 |
| | | Fréchet | 0.21*** | −0.09* | 0.28*** | 511 | 511 | 511 |
| | | N-D Fréchet | 0.16*** | −0.08 | 0.24*** | 511 | 511 | 511 |
| | | 1-D Hausdorff | 0.21*** | −0.09* | 0.28*** | 511 | 511 | 511 |
| | | N-D Hausdorff | 0.18*** | −0.09* | 0.18*** | 511 | 511 | 511 |
| | | AHV | −0.22*** | 0.11* | −0.35*** | 511 | 511 | 511 |
| | 12 | ADNS | −0.30*** | 0.21*** | −0.19*** | 418 | 423 | 452 |
| | | Euclidean | −0.27*** | 0.21*** | −0.16*** | 418 | 423 | 452 |
| | | 1-D Fréchet | −0.26*** | 0.20*** | −0.15** | 418 | 423 | 452 |
| | | N-D Fréchet | −0.20*** | 0.16** | −0.14** | 418 | 423 | 452 |
| | | 1-D Hausdorff | −0.26*** | 0.20*** | −0.15** | 418 | 423 | 452 |
| | | N-D Hausdorff | −0.13** | 0.06 | −0.06 | 418 | 423 | 452 |
| | | AHV | 0.23*** | −0.13** | 0.17*** | 418 | 423 | 452 |
| | 24 | ADNS | −0.39*** | 0.28*** | −0.34*** | 357 | 357 | 377 |
| | | Euclidean | −0.32*** | 0.20*** | −0.31*** | 357 | 357 | 377 |
| | | 1-D Fréchet | −0.31*** | 0.20*** | −0.31*** | 357 | 357 | 377 |
| | | N-D Fréchet | −0.27*** | 0.18*** | −0.26*** | 357 | 357 | 377 |
| | | 1-D Hausdorff | −0.31*** | 0.20*** | −0.31*** | 357 | 357 | 377 |
| | | N-D Hausdorff | −0.19*** | 0.14** | −0.20*** | 357 | 357 | 377 |
| | | AHV | 0.32*** | −0.15** | 0.25*** | 357 | 357 | 377 |
| | 36 | ADNS | −0.38*** | 0.24*** | −0.31*** | 284 | 273 | 309 |
| | | Euclidean | −0.33*** | 0.21*** | −0.25*** | 284 | 273 | 309 |
| | | 1-D Fréchet | −0.33*** | 0.22*** | −0.25*** | 284 | 273 | 309 |
| | | N-D Fréchet | −0.25*** | 0.15* | −0.16** | 284 | 273 | 309 |
| | | 1-D Hausdorff | −0.33*** | 0.22*** | −0.25*** | 284 | 273 | 309 |
| | | N-D Hausdorff | −0.19** | 0.09 | −0.10 | 284 | 273 | 309 |
| | | AHV | 0.35*** | −0.16** | 0.27*** | 284 | 273 | 309 |
| | 48 | ADNS | −0.42*** | 0.30*** | −0.31*** | 199 | 226 | 242 |
| | | Euclidean | −0.39*** | 0.22** | −0.25*** | 199 | 226 | 242 |
| | | 1-D Fréchet | −0.37*** | 0.22*** | −0.24*** | 199 | 226 | 242 |
| | | N-D Fréchet | −0.32*** | 0.16* | −0.18** | 199 | 226 | 242 |
| | | 1-D Hausdorff | −0.37*** | 0.22*** | −0.24*** | 199 | 226 | 242 |
| | | N-D Hausdorff | −0.27*** | 0.10 | −0.12 | 199 | 226 | 242 |
| | | AHV | 0.35*** | −0.19** | 0.28*** | 199 | 226 | 242 |
| AD | Baseline | ADNS | 0.27** | −0.17 | 0.33*** | 129 | 129 | 129 |
| | | Euclidean | 0.26** | −0.19* | 0.35*** | 129 | 129 | 129 |
| | | 1-D Fréchet | 0.27** | −0.18* | 0.37*** | 129 | 129 | 129 |
| | | N-D Fréchet | 0.28** | −0.12 | 0.34*** | 129 | 129 | 129 |
| | | 1-D Hausdorff | 0.27** | −0.18* | 0.37*** | 129 | 129 | 129 |
| | | N-D Hausdorff | 0.23* | −0.06 | 0.23** | 129 | 129 | 129 |
| | | AHV | −0.08 | 0.08 | −0.14 | 129 | 129 | 129 |
| | 12 | ADNS | 0.09 | 0.04 | 0.00 | 94 | 91 | 89 |
| | | Euclidean | 0.04 | 0.13 | −0.03 | 94 | 91 | 89 |
| | | 1-D Fréchet | 0.02 | 0.15 | −0.05 | 94 | 91 | 89 |
| | | N-D Fréchet | 0.01 | 0.01 | −0.06 | 94 | 91 | 89 |
| | | 1-D Hausdorff | 0.02 | 0.15 | −0.05 | 94 | 91 | 89 |
| | | N-D Hausdorff | 0.02 | −0.01 | 0.00 | 94 | 91 | 89 |
| | | AHV | −0.08 | −0.03 | −0.22* | 94 | 91 | 89 |
| | 24 | ADNS | 0.03 | 0.16 | −0.36 | 28 | 37 | 28 |
| | | Euclidean | −0.14 | 0.29 | −0.53** | 28 | 37 | 28 |
| | | 1-D Fréchet | −0.11 | 0.22 | −0.49** | 28 | 37 | 28 |
| | | N-D Fréchet | −0.17 | 0.01 | −0.36 | 28 | 37 | 28 |
| | | 1-D Hausdorff | −0.11 | 0.22 | −0.49** | 28 | 37 | 28 |
| | | N-D Hausdorff | −0.17 | −0.15 | −0.34 | 28 | 37 | 28 |
| | | AHV | −0.34 | −0.10 | −0.20 | 28 | 37 | 28 |
| | 36 | ADNS | — | −0.13 | — | 0 | 15 | 0 |
| | | Euclidean | — | −0.22 | — | 0 | 15 | 0 |
| | | 1-D Fréchet | — | −0.28 | — | 0 | 15 | 0 |
| | | N-D Fréchet | — | −0.48 | — | 0 | 15 | 0 |
| | | 1-D Hausdorff | — | −0.28 | — | 0 | 15 | 0 |
| | | N-D Hausdorff | — | −0.34 | — | 0 | 15 | 0 |
| | | AHV | — | −0.09 | — | 0 | 15 | 0 |
| | 48 | ADNS | — | 0.28 | — | 1 | 17 | 0 |
| | | Euclidean | — | −0.01 | — | 1 | 17 | 0 |
| | | 1-D Fréchet | — | 0.03 | — | 1 | 17 | 0 |
| | | N-D Fréchet | — | −0.07 | — | 1 | 17 | 0 |
| | | 1-D Hausdorff | — | 0.03 | — | 1 | 17 | 0 |
| | | N-D Hausdorff | — | −0.10 | — | 1 | 17 | 0 |

Table 9. Longitudinal Assessment of all Distance Metrics based on Diagnosis Transitions. Assessment of all distance metrics, benchmarked against AHV, on the basis of metric ability to predict progression, operationalized as two-tailed t-tests performed for each DTC comparison, z-scores, effect sizes (Cohen's d) with 95% CIs, and AUC-ROC with 95% CIs. Evaluation was performed in the full experimental cohort and in sub-groups based on starting diagnosis.

| Comparison | | t | Metric | AUC [95% CI] | Cohen's d [95% CI] | | z-score |
|---|---|---|---|---|---|---|---|
| Stable | All | 12 | ADNS | 0.67 [0.53, 0.80] | 0.64 | [0.35, 0.93]*** | −4.35 |
| vs | | | Euclidean | 0.65 [0.51, 0.79] | 0.52 | [0.23, 0.81]*** | −3.52 |
| Decline | | | 1-D Fréchet | 0.67 [0.49, 0.85] | 0.56 | [0.27, 0.85]*** | −3.81 |
| | | | N-D Fréchet | 0.61 [0.42, 0.79] | 0.46 | [0.17, 0.75]** | −3.15 |
| | | | 1-D Hausdorff | 0.65 [0.50, 0.80] | 0.56 | [0.27, 0.85]*** | −3.81 |
| | | | N-D Hausdorff | 0.62 [0.45, 0.78] | 0.32 | [0.03, 0.61]* | −2.20 |
| | | | AHV | 0.65 [0.50, 0.80] | −0.53 | [−0.81, −0.24]*** | −3.57 |
| | | 24 | ADNS | 0.71 [0.59, 0.82] | 0.84 | [0.61, 1.08]*** | −7.00 |
| | | | Euclidean | 0.67 [0.53, 0.80] | 0.66 | [0.42, 0.89]*** | −5.51 |
| | | | 1-D Fréchet | 0.67 [0.55, 0.79] | 0.69 | [0.45, 0.92]*** | −5.77 |
| | | | N-D Fréchet | 0.66 [0.54, 0.77] | 0.65 | [0.42, 0.89]*** | −5.49 |
| | | | 1-D Hausdorff | 0.67 [0.54, 0.80] | 0.69 | [0.45, 0.92]*** | −5.77 |
| | | | N-D Hausdorff | 0.65 [0.49, 0.81] | 0.65 | [0.42, 0.88]*** | −5.45 |
| | | | AHV | 0.71 [0.61, 0.82] | −0.79 | [−1.02, −0.55]*** | −6.58 |
| | | 36 | ADNS | 0.69 [0.53, 0.85] | 0.72 | [0.46, 0.98]*** | −5.42 |
| | | | Euclidean | 0.65 [0.51, 0.79] | 0.56 | [0.30, 0.82]*** | −4.23 |
| | | | 1-D Fréchet | 0.64 [0.52, 0.76] | 0.57 | [0.31, 0.83]*** | −4.33 |
| | | | N-D Fréchet | 0.62 [0.47, 0.77] | 0.43 | [0.17, 0.68]** | −3.24 |
| | | | 1-D Hausdorff | 0.64 [0.52, 0.77] | 0.57 | [0.31, 0.83]*** | −4.33 |
| | | | N-D Hausdorff | 0.62 [0.50, 0.74] | 0.46 | [0.20, 0.72]*** | −3.51 |
| | | | AHV | 0.72 [0.60, 0.85] | −0.79 | [−1.05, −0.52]*** | −5.88 |
| | | 48 | ADNS | 0.67 [0.52, 0.83] | 0.74 | [0.47, 1.01]*** | −5.36 |
| | | | Euclidean | 0.60 [0.43, 0.77] | 0.54 | [0.27, 0.81]*** | −3.92 |
| | | | 1-D Fréchet | 0.60 [0.43, 0.77] | 0.51 | [0.24, 0.78]*** | −3.71 |
| | | | N-D Fréchet | 0.60 [0.46, 0.73] | 0.45 | [0.18, 0.72]*** | −3.30 |
| | | | 1-D Hausdorff | 0.61 [0.47, 0.75] | 0.51 | [0.24, 0.78]*** | −3.71 |
| | | | N-D Hausdorff | 0.56 [0.42, 0.71] | 0.32 | [0.05, 0.59]* | −2.37 |
| | | | AHV | 0.72 [0.62, 0.83] | −0.79 | [−1.06, −0.51]*** | −5.66 |
| | CN | 12 | ADNS | 0.35 [0.08, 0.62] | −0.06 | [−0.70, 0.59] | −0.17 |
| | | | Euclidean | 0.39 [0.15, 0.63] | 0.05 | [−0.59, 0.69] | −0.16 |
| | | | 1-D Fréchet | 0.45 [0.09, 0.80] | 0.10 | [−0.54, 0.74] | −0.31 |
| | | | N-D Fréchet | 0.56 [0.17, 0.95] | 0.45 | [−0.20, 1.09] | −1.37 |
| | | | 1-D Hausdorff | 0.43 [0.14, 0.71] | 0.10 | [−0.54, 0.74] | −0.31 |
| | | | N-D Hausdorff | 0.66 [0.27, 1.04] | 0.54 | [−0.11, 1.18] | −1.64 |
| | | | AHV | 0.72 [0.35, 1.09] | −0.59 | [−1.23, 0.06] | −1.79 |
| | | 24 | ADNS | 0.64 [0.41, 0.88] | 0.55 | [0.08, 1.01]* | −2.32 |
| | | | Euclidean | 0.66 [0.42, 0.89] | 0.47 | [0.00, 0.93]* | −1.98 |
| | | | 1-D Fréchet | 0.69 [0.49, 0.89] | 0.51 | [0.04, 0.97]* | −2.15 |
| | | | N-D Fréchet | 0.75 [0.53, 0.96] | 0.75 | [0.28, 1.22]** | −3.17 |
| | | | 1-D Hausdorff | 0.63 [0.41, 0.85] | 0.51 | [0.04, 0.97]* | −2.15 |
| | | | N-D Hausdorff | 0.65 [0.38, 0.93] | 0.66 | [0.19, 1.13]** | −2.79 |
| | | | AHV | 0.74 [0.53, 0.95] | −0.79 | [−1.26, −0.33]*** | −3.34 |
| | | 36 | ADNS | 0.59 [0.21, 0.97] | 0.34 | [−0.36, 1.05] | −0.98 |
| | | | Euclidean | 0.44 [0.09, 0.80] | 0.07 | [−0.63, 0.77] | −0.21 |
| | | | 1-D Fréchet | 0.44 [0.07, 0.81] | 0.13 | [−0.57, 0.84] | −0.38 |
| | | | N-D Fréchet | 0.60 [0.24, 0.97] | 0.53 | [−0.18, 1.24] | −1.48 |
| | | | 1-D Hausdorff | 0.43 [0.05, 0.82] | 0.13 | [−0.57, 0.84] | −0.38 |
| | | | N-D Hausdorff | 0.70 [0.40, 1.00] | 0.58 | [−0.14, 1.29] | −1.62 |
| | | | AHV | 0.78 [0.46, 1.09] | −0.84 | [−1.57, −0.11]* | −2.30 |
| | | 48 | ADNS | 0.44 [0.14, 0.75] | 0.11 | [−0.44, 0.65] | −0.39 |
| | | | Euclidean | 0.40 [0.15, 0.65] | 0.04 | [−0.50, 0.58] | −0.15 |
| | | | 1-D Fréchet | 0.40 [0.15, 0.64] | 0.06 | [−0.48, 0.60] | −0.21 |
| | | | N-D Fréchet | 0.63 [0.34, 0.93] | 0.36 | [−0.18, 0.91] | −1.32 |
| | | | 1-D Hausdorff | 0.38 [0.10, 0.66] | 0.06 | [−0.48, 0.60] | −0.21 |
| | | | N-D Hausdorff | 0.40 [0.16, 0.63] | −0.04 | [−0.58, 0.50] | −0.14 |
| | | | AHV | 0.72 [0.46, 0.98] | −0.65 | [−1.20, −0.10]* | −2.34 |
| | MCI | 12 | ADNS | 0.77 [0.65, 0.90] | 1.08 | [0.75, 1.42]*** | −6.39 |
| | | | Euclidean | 0.73 [0.55, 0.90] | 1.00 | [0.67, 1.33]*** | −5.90 |
| | | | 1-D Fréchet | 0.73 [0.57, 0.89] | 0.97 | [0.64, 1.30]*** | −5.73 |
| | | | N-D Fréchet | 0.67 [0.49, 0.84] | 0.69 | [0.36, 1.02]*** | −4.13 |
| | | | 1-D Hausdorff | 0.72 [0.55, 0.89] | 0.97 | [0.64, 1.30]*** | −5.73 |
| | | | N-D Hausdorff | 0.64 [0.45, 0.83] | 0.45 | [0.12, 0.77]** | −2.69 |
| | | | AHV | 0.68 [0.51, 0.85] | −0.69 | [−1.02, −0.36]*** | −4.14 |
| | | 24 | ADNS | 0.76 [0.64, 0.88] | 1.05 | [0.77, 1.34]*** | −7.36 |
| | | | Euclidean | 0.68 [0.53, 0.83] | 0.84 | [0.56, 1.12]*** | −5.94 |
| | | | 1-D Fréchet | 0.69 [0.55, 0.84] | 0.82 | [0.54, 1.09]*** | −5.78 |
| | | | N-D Fréchet | 0.63 [0.47, 0.78] | 0.65 | [0.37, 0.92]*** | −4.61 |
| | | | 1-D Hausdorff | 0.70 [0.57, 0.83] | 0.82 | [0.54, 1.09]*** | −5.78 |
| | | | N-D Hausdorff | 0.68 [0.53, 0.84] | 0.66 | [0.39, 0.94]*** | −4.73 |
| | | | AHV | 0.73 [0.59, 0.86] | −0.85 | [−1.12, −0.57]*** | −5.99 |
| | | 36 | ADNS | 0.73 [0.62, 0.84] | 0.87 | [0.59, 1.16]*** | −5.99 |
| | | | Euclidean | 0.67 [0.50, 0.83] | 0.68 | [0.40, 0.97]*** | −4.74 |
| | | | 1-D Fréchet | 0.69 [0.54, 0.84] | 0.70 | [0.41, 0.98]*** | −4.82 |
| | | | N-D Fréchet | 0.63 [0.46, 0.81] | 0.44 | [0.16, 0.72]** | −3.06 |
| | | | 1-D Hausdorff | 0.69 [0.54, 0.84] | 0.70 | [0.41, 0.98]*** | −4.82 |
| | | | N-D Hausdorff | 0.63 [0.52, 0.73] | 0.49 | [0.21, 0.77]*** | −3.41 |
| | | | AHV | 0.73 [0.56, 0.90] | −0.86 | [−1.14, −0.57]*** | −5.87 |
| | | 48 | ADNS | 0.73 [0.57, 0.89] | 0.78 | [0.46, 1.10]*** | −4.83 |
| | | | Euclidean | 0.62 [0.42, 0.82] | 0.54 | [0.23, 0.86]*** | −3.41 |
| | | | 1-D Fréchet | 0.59 [0.44, 0.75] | 0.52 | [0.21, 0.83]** | −3.25 |
| | | | N-D Fréchet | 0.57 [0.41, 0.74] | 0.40 | [0.09, 0.71]* | −2.54 |
| | | | 1-D Hausdorff | 0.63 [0.44, 0.83] | 0.52 | [0.21, 0.83]** | −3.25 |
| | | | N-D Hausdorff | 0.60 [0.41, 0.78] | 0.36 | [0.05, 0.68]* | −2.30 |
| | | | AHV | 0.69 [0.51, 0.88] | −0.76 | [−1.08, −0.44]*** | −4.71 |

Table 10. Significant Neuroreader-Based ROIs by Z-Score Ranking and CN Template Values. The 12 significant regions extracted by performing ANOVA for each of the 22 Neuroreader-based regions in the ROI selection cohort are reported in the above table along with corresponding z-scores. Significance was established based on an alpha=0.05, Bonferroni-corrected. Structures are identified by anatomical labels used by Neuroreader. Mean volumes and standard deviations (SD) of the CN template cohort are included for each respective region.

| Neuroreader ROI Label | Z | CN Template Mean Vol (±SD) (mm³) |
|---|---|---|
| Left Hippocampus | 9.12 | 0.48 (±0.74) |
| Left Temporal Lobe | 8.43 | 0.45 (±0.68) |
| Left Amygdala | 8.43 | 0.60 (±0.79) |
| Right Amygdala | 8.37 | 0.54 (±0.80) |
| Right Hippocampus | 7.82 | 0.45 (±0.71) |
| Right Temporal Lobe | 7.53 | 0.44 (±0.72) |
| Left Parietal Lobe | 6.74 | 0.37 (±0.83) |
| Right Parietal Lobe | 6.55 | 0.40 (±0.93) |
| Left Frontal Lobe | 5.71 | 0.30 (±0.94) |
| Right Frontal Lobe | 5.48 | 0.26 (±0.98) |
| Left Thalamus | 5.17 | 0.33 (±1.01) |
| Right Thalamus | 4.42 | 0.31 (±1.02) |

Table 11. Baseline Assessment of all Distance Metrics based on Sensitivity to Diagnosis: Assessment of ADNeuro-Score on the basis of metric sensitivity to diagnosis at baseline, tested again using the Neuroreader-based ROIs to generate ADNeuro-Score. AHV, computed using the Neuroreader-based ROIs is included for benchmarking. Significant group comparisons are denoted by * to indicate p<0.05, ** to indicate p<0.01, and *** to indicate p<0.001, Holm-Bonferroni corrected.

| Group | | | | z- |
|---|---|---|---|---|
| Comparison | Metric | AUC [95% CI] | Cohen's d [95% CI] | score |
| CN vs MCI | ADNS | 0.64 [0.57, 0.71] | −0.55 [−0.58, −0.52]*** | −7.31 |
| | AHV | 0.65 [0.56, 0.74] | 0.55 [0.52, 0.58]*** | −7.27 |
| CN vs AD | ADNS | 0.91 [0.86, 0.97] | −2.11 [−2.29, −1.93]*** | −16.67 |
| | AHV | 0.89 [0.82, 0.95] | 1.72 [1.55, 1.89]*** | −14.27 |
| MCI vs AD | ADNS | 0.81 [0.74, 0.88] | −1.18 [−1.20, −1.15]*** | −11.35 |
| | AHV | 0.76 [0.67, 0.85] | 0.92 [0.90, 0.94]*** | −9.04 |

Table 12. Linear Regression Results using Neuroreader-Based ROIs. Repeated baseline and longitudinal assessment of AD-NeuroScore based on metric association with disease severity (MMSE, ADAS-11, and CDR-SB scores) along with the AHV benchmark, tested with linear regression in the overall experimental cohort and in each diagnostic sub-group, using the Neuroreader-based ROIs to generate AD-NeuroScore and AHV. Significant group comparisons are denoted by * to indicate p<0.05, ** to indicate p<0.01, and *** to indicate p<0.001, Holm-Bonferroni corrected.

| | | | r | | | n | | |
|---|---|---|---|---|---|---|---|---|
| Class | t | Metric | CDR-SB | MMSE | ADAS-11 | CDR-SB | MMSE | ADAS-11 |
| All | Baseline | ADNeuro-Score | | | | | | |
| | | AHV | −0.43*** | 0.28*** | −0.47*** | 926 | 926 | 926 |
| | 12 | ADNeuro-Score | −0.27*** | 0.20*** | −0.24*** | 525 | 697 | 721 |
| | | AHV | 0.23*** | −0.16*** | 0.19*** | 525 | 697 | 721 |
| | 24 | ADNeuro-Score | −0.40*** | 0.26*** | −0.38*** | 402 | 614 | 626 |
| | | AHV | 0.32*** | −0.19*** | 0.26*** | 402 | 614 | 626 |
| | 36 | ADNeuro-Score | −0.39*** | 0.21*** | −0.31*** | 285 | 332 | 345 |
| | | AHV | 0.34*** | −0.17** | 0.26*** | 285 | 332 | 345 |
| | 48 | ADNeuro-Score | −0.42*** | 0.25*** | −0.31*** | 205 | 369 | 375 |
| | | AHV | 0.35*** | −0.19*** | 0.26*** | 205 | 369 | 375 |
| CN | Baseline | ADNeuro-Score | −0.07 | −0.01 | 0.08 | 286 | 286 | 286 |
| | | AHV | 0.00 | −0.01 | −0.17** | 286 | 286 | 286 |
| | 12 | ADNeuro-Score | −0.74*** | −0.05 | 0.04 | 13 | 183 | 180 |
| | | AHV | 0.53 | −0.03 | 0.01 | 13 | 183 | 180 |
| | 24 | ADNeuro-Score | −0.57** | −0.07 | −0.07 | 17 | 220 | 221 |
| | | AHV | 0.45 | −0.08 | 0.05 | 17 | 220 | 221 |
| | 36 | ADNeuro-Score | — | −0.07 | −0.02 | 1 | 44 | 36 |
| | | AHV | — | −0.19 | 0.03 | 1 | 44 | 36 |
| | 48 | ADNeuro-Score | −0.11 | 0.03 | −0.07 | 5 | 126 | 133 |
| | | AHV | 0.52 | −0.21* | −0.01 | 5 | 126 | 133 |
| MCI | Baseline | ADNeuro-Score | 0.26*** | −0.10* | 0.32*** | 511 | 511 | 511 |
| | | AHV | −0.22*** | 0.11* | −0.35*** | 511 | 511 | 511 |
| | 12 | ADNeuro-Score | −0.29*** | 0.20*** | −0.20*** | 418 | 423 | 452 |
| | | AHV | 0.23*** | −0.13** | 0.17*** | 418 | 423 | 452 |
| | 24 | ADNeuro-Score | −0.40*** | 0.28*** | −0.34*** | 357 | 357 | 377 |
| | | AHV | 0.32*** | −0.15** | 0.25*** | 357 | 357 | 377 |
| | 36 | ADNeuro-Score | −0.39*** | 0.23*** | −0.31*** | 284 | 273 | 309 |
| | | AHV | 0.35*** | −0.16** | 0.27*** | 284 | 273 | 309 |
| | 48 | ADNeuro-Score | −0.42*** | 0.29*** | −0.31*** | 199 | 226 | 242 |
| | | AHV | 0.35*** | −0.19** | 0.28*** | 199 | 226 | 242 |
| AD | Baseline | ADNeuro-Score | 0.23** | −0.15 | 0.29*** | 129 | 129 | 129 |
| | | AHV | −0.08 | 0.08 | −0.14 | 129 | 129 | 129 |
| | 12 | ADNeuro-Score | 0.09 | 0.01 | 0.02 | 94 | 91 | 89 |
| | | AHV | −0.08 | −0.03 | −0.22* | 94 | 91 | 89 |
| | 24 | ADNeuro-Score | 0.11 | 0.14 | −0.28 | 28 | 37 | 28 |
| | | AHV | −0.34 | −0.10 | −0.20 | 28 | 37 | 28 |
| | 36 | ADNeuro-Score | — | −0.06 | — | 0 | 15 | 0 |
| | | AHV | — | −0.09 | — | 0 | 15 | 0 |
| | 48 | ADNeuro-Score | — | 0.26 | — | 1 | 17 | 0 |
| | | AHV | — | −0.11 | — | 1 | 17 | 0 |

Table 13. Longitudinal Assessment of AD-NeuroScore (ADNS) and AHV based on Diagnosis Transitions Using Neuroreader-Based ROIs. AD-NeuroScore (ADNS) assessment based on ability to predict progression (DTC) at each longitudinal session, using Neuroreader analogous ROIs. Results are stratified by diagnostic group. Two-tailed t-tests were performed for each DTC comparison and z-scores, effect sizes (Cohen's d) with 95% CIs, and AUC-ROC values with 95% confidence intervals were subsequently calculated. Significant group comparisons are denoted by * to indicate p<0.05, ** to indicate p<0.01, and *** to indicate

| Comparison | | t | Metric | AUC [95% CI] | Cohen's d [95% CI] | | z-score |
|---|---|---|---|---|---|---|---|
| Stable | All | 12 months | ADNS | 0.66 [0.50, 0.83] | 0.62 | [0.33, 0.91]*** | −4.21 |
| vs | | | AHV | 0.66 [0.53, 0.79] | −0.53 | [−0.81, −0.24]*** | −3.57 |
| Decline | | 24 months | ADNS | 0.72 [0.58, 0.86] | 0.86 | [0.62, 1.09]*** | −7.14 |
| | | | AHV | 0.72 [0.62, 0.83] | −0.79 | [−1.02, −0.55]*** | −6.58 |
| | | 36 months | ADNS | 0.69 [0.58, 0.81] | 0.74 | [0.48, 1.00]*** | −5.53 |
| | | | AHV | 0.72 [0.59, 0.84] | −0.79 | [−1.05, −0.52]*** | −5.88 |
| | | 48 months | ADNS | 0.68 [0.55, 0.82] | 0.77 | [0.49, 1.04]*** | −5.53 |
| | | | AHV | 0.71 [0.58, 0.85] | −0.79 | [−1.06, −0.51]*** | −5.66 |
| | CN | 12 months | ADNS | 0.54 [0.14, 0.94] | −0.24 | [−0.88, 0.40] | −0.73 |
| | | | AHV | 0.65 [0.27, 1.03] | −0.59 | [−1.23, 0.06] | −1.79 |
| | | 24 months | ADNS | 0.56 [0.22, 0.89] | 0.36 | [−0.10, 0.82] | −1.53 |
| | | | AHV | 0.75 [0.51, 0.99] | −0.79 | [−1.26, −0.33]*** | −3.34 |
| | | 36 months | ADNS | 0.34 [0.06, 0.62] | −0.02 | [−0.72, 0.68] | −0.06 |
| | | | AHV | 0.77 [0.48, 1.07] | −0.84 | [−1.57, −0.11]* | −2.30 |
| | | 48 months | ADNS | 0.43 [0.09, 0.77] | 0.11 | [−0.44, 0.65] | −0.39 |
| | | | AHV | 0.73 [0.51, 0.94] | −0.65 | [−1.20, −0.10]* | −2.34 |
| | MCI | 12 months | ADNS | 0.77 [0.63, 0.91] | 1.07 | [0.74, 1.41]*** | −6.33 |
| | | | AHV | 0.69 [0.51, 0.87] | −0.69 | [−1.02, −0.36]*** | −4.14 |
| | | 24 months | ADNS | 0.77 [0.65, 0.88] | 1.12 | [0.84, 1.40]*** | −7.78 |
| | | | AHV | 0.72 [0.59, 0.85] | −0.85 | [−1.12, −0.57]*** | −5.99 |
| | | 36 months | ADNS | 0.73 [0.61, 0.86] | 0.94 | [0.65, 1.23]*** | −6.41 |
| | | | AHV | 0.74 [0.61, 0.86] | −0.86 | [−1.14, −0.57]*** | −5.87 |
| | | 48 months | ADNS | 0.73 [0.57, 0.89] | 0.82 | [0.51, 1.14]*** | −5.09 |

| | | | |
|---|---|---|---|
| AHV | 0.69 [0.53, 0.86] | −0.76 p−1.08, −0.44]*** | −4.71 |

p<0.001, Holm-Bonferroni corrected.

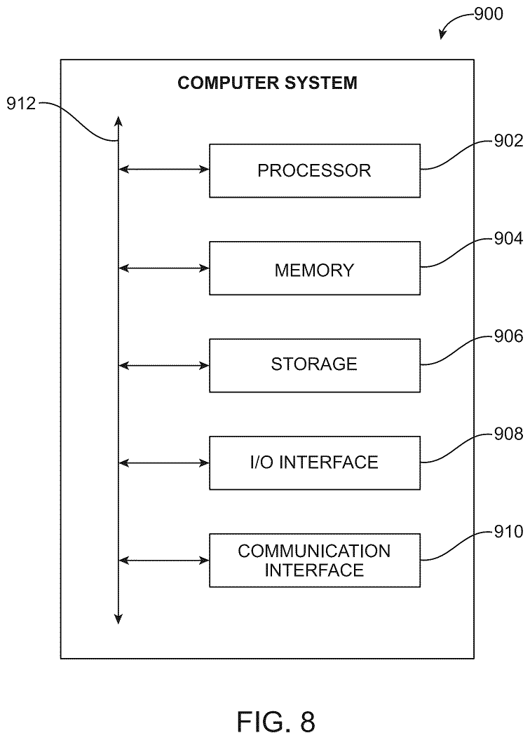

FIG. 8 illustrates an example computer system 800. In particular exemplary implementations, one or more computer systems 800 perform one or more steps of one or more methods described or illustrated herein. In particular exemplary implementations, one or more computer systems 800 provide functionality described or illustrated herein. In particular exemplary implementations, software running on one or more computer systems 800 performs one or more steps of one or more methods described or illustrated herein or provides functionality described or illustrated herein. Particular implementations may include one or more portions of one or more computer systems 800. Herein, reference to a computer system may encompass a computing device, and vice versa, where appropriate. Moreover, reference to a computer system may encompass one or more computer systems, where appropriate.

This disclosure contemplates any suitable number of computer systems 800. This disclosure contemplates computer system 800 taking any suitable physical form. As example and not by way of limitation, computer system 800 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, computer system 800 may include one or more computer systems 800; be unitary or distributed; span multiple locations; span multiple machines; span multiple data centers; or reside in a cloud, which may include one or more cloud components in one or more networks. Where appropriate, one or more computer systems 800 may perform without substantial spatial or temporal limitation one or more steps of one or more methods described or illustrated herein. As an example and not by way of limitation, one or more computer systems 800 may perform in real time or in batch mode one or more steps of one or more methods described or illustrated herein. One or more computer systems 800 may perform at different times or at different locations one or more steps of one or more methods described or illustrated herein, where appropriate.

In particular exemplary implementations, computer system 800 includes a processor 802, memory 804, storage 806, an input/output (I/O) interface 808, a communication interface 810, and a bus 812. Although this disclosure describes and illustrates a particular computer system having a particular number of particular components in a particular arrangement, this disclosure contemplates any suitable computer system having any suitable number of any suitable components in any suitable arrangement.

In particular exemplary implementations, processor 802 includes hardware for executing instructions, such as those making up a computer program. As an example and not by way of limitation, to execute instructions, processor 802 may retrieve (or fetch) the instructions from an internal register, an internal cache, memory 804, or storage 806; decode and execute them; and then write one or more results to an internal register, an internal cache, memory 804, or storage 806. In particular exemplary implementations, processor 802 may include one or more internal caches for data, instructions, or addresses. This disclosure contemplates processor 802 including any suitable number of any suitable internal caches, where appropriate. As an example and not by way of limitation, processor 802 may include one or more instruction caches, one or more data caches, and one or more translation lookaside buffers (TLBs). Instructions in the instruction caches may be copies of instructions in memory 804 or storage 806, and the instruction caches may speed up retrieval of those instructions by processor 802. Data in the data caches may be copies of data in memory 804 or storage 806 for instructions executing at processor 802 to operate on; the results of previous instructions executed at processor 802 for access by subsequent instructions executing at processor 802 or for writing to memory 804 or storage 806; or other suitable data. The data caches may speed up read or write operations by processor 802. The TLBs may speed up virtual-address translation for processor 802. In particular exemplary implementations, processor 802 may include one or more internal registers for data, instructions, or addresses. This disclosure contemplates processor 802 including any suitable number of any suitable internal registers, where appropriate. Where appropriate, processor 802 may include one or more arithmetic logic units (ALUs); be a multi-core processor; or include one or more processors 802. Although this disclosure describes and illustrates a particular processor, this disclosure contemplates any suitable processor.

In particular exemplary implementations, memory 804 includes main memory for storing instructions for processor 802 to execute or data for processor 802 to operate on. As an example and not by way of limitation, computer system 800 may load instructions from storage 806 or another source (such as, for example, another computer system 800) to memory 804. Processor 802 may then load the instructions from memory 804 to an internal register or internal cache. To execute the instructions, processor 802 may retrieve the instructions from the internal register or internal cache and decode them. During or after execution of the instructions, processor 802 may write one or more results (which may be intermediate or final results) to the internal register or internal cache. Processor 802 may then write one or more of those results to memory 804. In particular exemplary implementations, processor 802 may execute instructions in one or more internal registers or internal caches or in memory 804 (as opposed to storage 806 or elsewhere) and operates on data in one or more internal registers or internal caches or in memory 804 (as opposed to storage 806 or elsewhere). One or more memory buses (which may each include an address bus and a data bus) may couple processor 802 to memory 804. Bus 812 may include one or more memory buses, as described below. In particular exemplary implementations, one or more memory management units (MMUs) may reside between processor 802 and memory 804 and may facilitate accesses to memory 804 requested by processor 802. In particular exemplary implementations, memory 804 includes random access memory (RAM). This RAM may be volatile memory, where appropriate Where appropriate, this RAM may be dynamic RAM (DRAM) or static RAM (SRAM). Moreover, where appropriate, this RAM may be single-ported or multi-ported RAM. This disclosure contemplates any suitable RAM. Memory 804 may include one or more memories 804, where appropriate. Although this disclosure describes and illustrates particular memory, this disclosure contemplates any suitable memory.

In particular exemplary implementations, storage 806 includes mass storage for data or instructions. As an example and not by way of limitation, storage 806 may include a hard disk drive (HDD), a floppy disk drive, flash memory, an optical disc, a magneto-optical disc, magnetic tape, or a Universal Serial Bus (USB) drive or a combination of two or more of these. Storage 806 may include removable or non-removable (or fixed) media, where appropriate. Storage 806 may be internal or external to computer system 800, where appropriate. In particular exemplary implementations, storage 806 is non-volatile, solid-state memory. In particular exemplary implementations, storage 806 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 806 taking any suitable physical form. Storage 806 may include one or more storage control units facilitating communication between processor 802 and storage 806, where appropriate. Where appropriate, storage 806 may include one or more storages 806. Although this disclosure describes and illustrates particular storage, this disclosure contemplates any suitable storage.

In particular exemplary implementations, I/O interface 808 includes hardware, software, or both, providing one or more interfaces for communication between computer system 800 and one or more I/O devices. Computer system 800 may include one or more of these I/O devices, where appropriate. One or more of these I/O devices may enable communication between a person and computer system 800. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 808 for them. Where appropriate, I/O interface 808 may include one or more device or software drivers enabling processor 802 to drive one or more of these I/O devices. I/O interface 808 may include one or more I/O interfaces 808, where appropriate. Although this disclosure describes and illustrates a particular I/O interface, this disclosure contemplates any suitable I/O interface.

In particular exemplary implementations, communication interface 810 includes hardware, software, or both providing one or more interfaces for communication (such as, for example, packet-based communication) between computer system 800 and one or more other computer systems 800 or one or more networks. As an example and not by way of limitation, communication interface 810 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 810 for it. As an example and not by way of limitation, computer system 800 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 800 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 800 may include any suitable communication interface 810 for any of these networks, where appropriate. Communication interface 810 may include one or more communication interfaces 810, where appropriate. Although this disclosure describes and illustrates a particular communication interface, this disclosure contemplates any suitable communication interface.

In particular exemplary implementations, bus 812 includes hardware, software, or both coupling components of computer system 800 to each other. As an example and not by way of limitation, bus 812 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 812 may include one or more buses 812, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

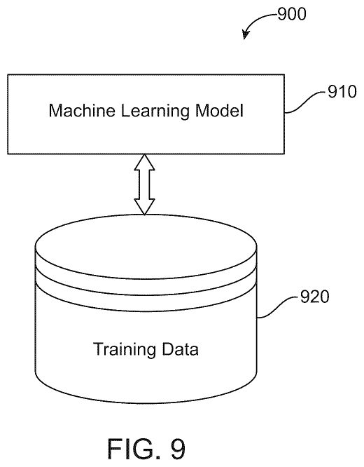

FIG. 9 illustrates a framework 900 employed by a software application (e.g., algorithm) for evaluating attributes of a gesture. The framework 900 may be hosted remotely. Alternatively, the framework 900 may reside within a storage and/or be processed by the computing system 800 shown in FIG. 8. The machine learning model 910 is operably coupled to the stored training data in a database 920.

In an exemplary implementation, the training data 920 may include attributes of thousands of objects. For example, the object may be MR images, brain scans, brain regions, biomarker metric, indications of cognitive states, and other cohorts of data. Attributes may include but are not limited to the size, shape, orientation, position of an object, brain region, etc. The training data 920 employed by the machine learning model 910 may be fixed or updated periodically. Alternatively, the training data 920 may be updated in real-time based upon the evaluations performed by the machine learning model 910 in a non-training mode. This is illustrated by the double-sided arrow connecting the machine learning model 910 and stored training data 920.

In operation, the machine learning model 910 may evaluate attributes of images/videos obtained by hardware (see, e.g., FIG. 8). The attributes of the captured image (e.g., captured MR image) are then compared with respective attributes of stored training data 920 (e.g., prestored training images, and MR images). The likelihood of similarity between each of the obtained attributes (e.g., of the captured image of a brain region) and the stored training data 920 (e.g., prestored and/or pre-analyzed MR images) is given a confidence score. In one exemplary implementation, if the confidence score exceeds a predetermined threshold, the attribute is included in an image description (e.g., a region or volume of interest, etc.) that is ultimately communicated to the user via a user interface of a computing device. In another exemplary implementation, the description may include a certain number of attributes which exceed a predetermined threshold to share with the user. The sensitivity of sharing more or less attributes can be customized based upon the needs of the particular user.

In addition to the benefits described above, the examples of the present disclosure facilitate identification of biomarkers and other indications for a neurodegenerative disease A main challenge in doing so is determining how to ensure that an extracted representation is indeed relevant to a brain region or other imaging associated with an individual.

Typically, such determinations require a large quantity of manual analysis, observation, and annotation to obtain and analyze the training data in a supervised training framework. However, aspects of the present disclosure, including the MR image assessment, brain region recognition, benchmark determinations, biomarker metrics, cognitive states, and assessment scores may deploy a machine learning model that is flexible, adaptive, automated, and trainable. Manual operations need not be necessary due to a self-supervised learning framework and set-embedding neural network model design. As such, this enables the vibe signature to be flexible and scalable.

Moreover, examples of the present disclosure may utilize automated machine learning models, in part, to achieve the goal of analyzing disparate forms, types, and features of data and information, which may be received from a variety of computing devices and other sources. Such disparate forms of data may be indicative of MR images, other medical images, neurodegenerative disease information, including biomarker metrics, distance metrics, assessment scores, scores associated with cognitive states, cohorts of data, and the like.

The benchmarks and assessment scores may provide a common format which can be compared, contrasted, and analyzed, thus enabling diverse types of online content to be analyzed, reviewed, and/or utilized. Such techniques may significantly improve efficiencies with respect to review, processing, and analysis, reducing and/or eliminating a need for manual review or subjective determinations, and providing techniques for automation. For example, the automated machine learning models may be trained, based on input training data associated with identifying of different types of MR images, medical images, neurodegenerative disease data, benchmarks associated with certain states of neurodegenerative disease, biomarkers and biomarker metrics associated with the same, assessment scores associated with benchmarks, among others. Automated machine learning models may be provided inputs of different weights and values, to enable customization for certain features and aspects which may be of interest. In this regard, the automated machine learning models are configured to (automatically) analyze, extract, and identify content items and features in an efficient, streamlined manner, and generate a common format, e.g., brain region volumes, biomarkers, assessment scores, cognitive states, etc., to enable comparisons between disparate features, content, and aspects. As such, individuals and devices need not analyze content in a computationally intensive, e.g., brute force manner, as may exist in some conventional systems, and inefficiently drains and constrains process resources and capacity of devices. Additionally, based on the automated machine learning models' ability to quickly learn and predict features, items, styles, etc., over time, the accuracy of the predictions of content violations may be significantly increased and thereby significantly improve (e.g., minimizes) the network latency associated with the network and may thus minimize network traffic across the network by reducing the amount of content/data traffic associated with the network.

Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another implementation includes from the one particular value and/or to the other particular value. Similarly, when values are expressed as approximations, by use of the antecedent “about,” it will be understood that the particular value forms another implementation. All ranges are inclusive and combinable, and it should be understood that steps may be performed in any order.

It is to be appreciated that certain features of the invention which are, for clarity, described herein in the context of separate implementations, may also be provided in combination in a single implementation. Conversely, various features of the invention that are, for brevity, described in the context of a single implementation, may also be provided separately or in any subcombination. All documents cited herein are incorporated herein in their entireties for any and all purposes.

Further, reference to values stated in ranges include each and every value within that range. In addition, the term “comprising” should be understood as having its standard, open-ended meaning, but also as encompassing “consisting” as well. For example, a device that comprises Part A and Part B may include parts in addition to Part A and Part B, but may also be formed only from Part A and Part B.

The present invention has been demonstrated to outperform current commercial software tools for optimizing and simulating lattice structures, as demonstrated in Appendix A.

### REFERENCES

Achterberg, H. C., van der Lijn, F., den Heijer, T., Vernooij, M. W., Ikram, M. A., Niessen, W. J., de Bruijne, M., 2014. Hippocampal shape is predictive for the development of dementia in a normal, elderly population. Human Brain Mapping 35, 2359-2371. https://doi.org/10.1002/HBM.22333
Ahdidan, J., Raji, C. A., De Yoe, E. A., Mathis, J., Noe, K. O., Rimestad, J., Kjeldsen, T. K., Mosegaard, J., Becker, J. T., Lopez, O., 2016. Quantitative Neuroimaging Software for Clinical Assessment of Hippocampal Volumes on MR Imaging. J Alzheimers Dis 49, 723-732. https://doi.org/10.3233/JAD-150559
Arimura, H., Yoshiura, T., Kumazawa, S., Tanaka, K., Koga, H., Mihara, F., Honda, H., Sakai, S., Toyofuku, F., Higashida, Y., 2008. Automated Method for Identification of Patients With Alzheimer's Disease Based on Three-dimensional MR Images. Academic Radiology 15, 274-284. https://doi.org/10.1016/j.acra.2007.10.020
Attier-Zmudka, J., Sérot, J. M., Valluy, J., Saffarini, M., Macaret, A. S., Diouf, M., Dao, S., Douadi, Y., Piotr Malinowski, K., Balédent, O., 2019. Decreased Cerebrospinal Fluid Flow Is Associated With Cognitive Deficit in Elderly Patients. Frontiers in Aging Neuroscience 11. https://doi.org/10.3389/FNAGI.2019.00087
Balsis, S., Benge, J. F., Lowe, D. A., Geraci, L., Doody, R. S., 2015. How Do Scores on the ADAS-Cog, MMSE, and CDR-SOB Correspond? Clin Neuropsychol 29, 1002-1009. https://doi.org/10.1080/13854046.2015.1119312
Braak, H., Braak, Eva, Braak, (H, Braak, E, 1997. Staging of Alzheimer-Related Cortical Destruction. International Psychogeriatrics 9, 257-261. https://doi.org/10.1017/S1041610297004973
Brewer, J. B., Magda, S., Airriess, C., Smith, M. E., 2009. Fully-Automated Quantification of Regional Brain Volumes for Improved Detection of Focal Atrophy in Alzheimer Disease. American Journal of Neuroradiology 30, 578-580. https://doi.org/10.3174/AJNR.A1402
Cabeza, R., Ciaramelli, E., Olson, I. R., Moscovitch, M., 2008. Parietal Cortex and Episodic Memory: An Attentional Account. Nat Rev Neurosci 9, 613. https://doi.org/10.1038/NRN2459
Casanova, R., Barnard, R. T., Gaussoin, S. A., Saldana, S., Hayden, K. M., Manson, J. A. E., Wallace, R. B., Rapp, S. R., Resnick, S. M., Espeland, M. A., Chen, J. C., 2018a. Using high-dimensional machine learning methods to estimate an anatomical risk factor for Alzheimer's disease across imaging databases. Neuroimage 183, 401-411. https://doi.org/10.1016/J.NEUROIMAGE.2018.08.040
Casanova, R., Barnard, R. T., Gaussoin, S. A., Saldana, S., Hayden, K. M., Manson, J. A. E., Wallace, R. B., Rapp, S. R., Resnick, S. M., Espeland, M. A., Chen, J. C., 2018b. Using high-dimensional machine learning methods to estimate an anatomical risk factor for Alzheimer's disease across imaging databases. Neuroimage 183, 401-411. https://doi.org/10.1016/J.NEUROIMAGE.2018.08.040
Casanova, R., Hsu, F. C., Sink, K. M., Rapp, S. R., Williamson, J. D., Resnick, S. M., Espeland, M. A., 2013. Alzheimer's Disease Risk Assessment Using Large-Scale Machine Learning Methods. PLOS ONE 8, e77949. https://doi.org/10.1371/JOURNAL.PONE.0077949
Cavedo, E., Tran, P., Thoprakarn, U., Martini, J. B., Movschin, A., Delmaire, C., Gariel, F., Heidelberg, D., Pyatigorskaya, N., Ströer, S., Krolak-Salmon, P., Cotton, F., dos Santos, C. L., Dormont, D., 2022. Validation of an automatic tool for the rapid measurement of brain atrophy and white matter hyperintensity: QyScore®. Eur Radiol 32, 2949-2961. https://doi.org/10.1007/S00330-021-08385-9
Chow, N., Hwang, K. S., Hurtz, S., Green, A. E., Somme, J. H., Thompson, P. M., Elashoff, D. A., Jack, C. R., Weiner, M., Apostolova, L. G., 2015. Comparing 3T and 1.5T MRI for Mapping Hippocampal Atrophy in the Alzheimer's Disease Neuroimaging Initiative. American Journal of Neuroradiology 36, 653-660. https://doi.org/10.3174/AJNR.A4228
Chung, J. K., Jang, J.-W., Initiative, the A.D.N., 2021. Comprehensive Visual Rating Scale on Magnetic Resonance Imaging: Application to Prodromal Alzheimer Disease. Annals of Geriatric Medicine and Research 25, 39. https://doi.org/10.4235/AGMR.21.0010
Coupé, P., Fonov, V. S., Bernard, C., Zandifar, A., Eskildsen, S. F., Helmer, C., Manjón, J. v., Amieva, H., Dartigues, J. F., Allard, M., Catheline, G., Collins, D. L., 2015a. Detection of Alzheimer's disease signature in MR images seven years before conversion to dementia: Toward an early individual prognosis. Hum Brain Mapp 36, 4758-4770. https://doi.org/10.1002/HBM.22926
Coupé, P., Fonov, V. S., Bernard, C., Zandifar, A., Eskildsen, S. F., Helmer, C., Manjón, J. v., Amieva, H., Dartigues, J. F., Allard, M., Catheline, G., Collins, D. L., 2015b. Detection of Alzheimer's disease signature in MR images seven years before conversion to dementia: Toward an early individual prognosis. Hum Brain Mapp 36, 4758-4770. https://doi.org/10.1002/HBM.22926
Coupé, P., Fonov, V. S., Bernard, C., Zandifar, A., Eskildsen, S. F., Helmer, C., Manjón, J. v., Amieva, H., Dartigues, J. F., Allard, M., Catheline, G., Collins, D. L., 2015c. Detection of Alzheimer's disease signature in MR images seven years before conversion to dementia: Toward an early individual prognosis. Human Brain Mapping 36, 4758-4770. https://doi.org/10.1002/HBM.22926
Coupé, P., Fonov, V. S., Bernard, C., Zandifar, A., Eskildsen, S. F., Helmer, C., Manjón, J. v, Amieva, H., Dartigues, J. F., Allard, M., Catheline, G., Collins, D. L., 2015d. Detection of Alzheimer's disease signature in MR images seven years before conversion to dementia: Toward an early individual prognosis. Human Brain Mapping 36, 4758-4770. https://doi.org/10.1002/HBM.22926
Coupé, P., Fonov, V. S., Bernard, C., Zandifar, A., Eskildsen, S. F., Helmer, C., Manjón, J. v., Amieva, H., Dartigues, J. F., Allard, M., Catheline, G., Collins, D. L., 2015e. Detection of Alzheimer's disease signature in MR images seven years before conversion to dementia: Toward an early individual prognosis. Human Brain Mapping 36, 4758-4770. https://doi.org/10.1002/HBM.22926
Csernansky, J. G., Wang, L., Joshi, S. C., Tilak Ratnanather, J., Miller, M. I., 2004. Computational anatomy and neuropsychiatric disease: probabilistic assessment of variation and statistical inference of group difference, hemispheric asymmetry, and time-dependent change. Neuroimage 23, S56-S68. https://doi.org/10.1016/J.NEUROIMAGE.2004.07.025
Dale, A., Fischl, B., Sereno, M. I., 1999. Cortical Surface-Based Analysis: I. Segmentation and Surface Reconstruction. Neuroimage 9, 179-194.
Desikan, R. S., Ségonne, F., Fischl, B., Quinn, B. T., Dickerson, B. C., Blacker, D., Buckner, R. L., Dale, A. M., Maguire, R. P., Hyman, B. T., Albert, M. S., Killiany, R. J., 2006. An automated labeling system for subdividing the human cerebral cortex on MRI scans into gyral based regions of interest. Neuroimage 31, 968-980. https://doi.org/DOI: 10.1016/j.neuroimage.2006.01.021
Diciotti, S., Ginestroni, A., Bessi, V., Giannelli, M., Tessa, C., Bracco, L., Mascalchi, M., Toschi, N., 2012a. Identification of mild Alzheimer's disease through automated classification of structural MRI features. Annu Int Conf IEEE Eng Med Biol Soc 2012, 428-431. https://doi.org/10.1109/EMBC.2012.6345959
Diciotti, S., Ginestroni, A., Bessi, V., Giannelli, M., Tessa, C., Bracco, L., Mascalchi, M., Toschi, N., 2012b. Identification of mild Alzheimer's Disease through automated classification of structural MRI features. Proceedings of the Annual International Conference of the IEEE Engineering in Medicine and Biology Society, EMBS 428-431. https://doi.org/10.1109/EMBC.2012.6345959
Dickerson, B. C., Goncharova, I., Sullivan, M. P., Forchetti, C., Wilson, R. S., Bennett, D. A., Beckett, L. A., DeToledo-Morrell, L., 2001. MRI-derived entorhinal and hippocampal atrophy in incipient and very mild Alzheimer's disease. Neurobiol Aging 22, 747-754. https://doi.org/10.1016/S0197-4580 (01) 00271-8
Dukart, J., Mueller, K., Barthel, H., Villringer, A., Sabri, O., Schroeter, M. L., 2013. Meta-analysis based SVM classification enables accurate detection of Alzheimer's disease across different clinical centers using FDG-PET and MRI. Psychiatry Res 212, 230-236. https://doi.org/10.1016/J.PSCYCHRESNS.2012.04.007
Dumitrescu, A., Rote, G., 2004. On the Fréchet distance of a set of curves.
Elahi, S., Bachman, A. H., Lee, S. H., Sidtis, J. J., Ardekani, B. A., 2015. Corpus Callosum Atrophy Rate in Mild Cognitive Impairment and Prodromal Alzheimer's Disease. J Alzheimers Dis 45, 921. https://doi.org/10.3233/JAD-142631
Fischl, B., Liu, A., Dale, A.~M., 2001. Automated manifold surgery: constructing geometrically accurate and topologically correct models of the human cerebral cortex. IEEE Medical Imaging 20, 70-80.
Fischl, B., Salat, D.~H., Busa, E., Albert, M., Dieterich, M., Haselgrove, C., van der Kouwe, A., Killiany, R., Kennedy, D., Klaveness, S., Montillo, A., Makris, N., Rosen, B., Dale, A.~M., 2002. Whole brain segmentation: automated labeling of neuroanatomical structures in the human brain. Neuron 33, 341-355.
Fischl, B., Salat, D. H., van der Kouwe, A. J. W., Makris, N., Ségonne, F., Quinn, B. T., Dale, A. M., 2004a. Sequence-independent segmentation of magnetic resonance images. Neuroimage 23, S69-S84. https://doi.org/DOI: 10.1016/j.neuroimage.2004.07.016
Fischl, B., Sereno, M. I., Dale, A., 1999a. Cortical Surface-Based Analysis: II: Inflation, Flattening, and a Surface-Based Coordinate System. Neuroimage 9, 195-207.
Fischl, B., Sereno, M. I., Tootell, R. B. H., Dale, A. M., 1999b. High-resolution intersubject averaging and a coordinate system for the cortical surface. Human Brain Mapping 8, 272-284. https://doi.org/10.1002/(SICI) 1097-0193 (1999) 8: 4<272:AID-HBM10>3.0. CO; 2-4
Fischl, B., van der Kouwe, A., Destrieux, C., Halgren, E., Ségonne, F., Salat, D. H., Busa, E., Seidman, L. J., Goldstein, J., Kennedy, D., Caviness, V., Makris, N., Rosen, B., Dale, A. M., 2004b. Automatically Parcellating the Human Cerebral Cortex. Cerebral Cortex 14, 11-22. https://doi.org/10.1093/cercor/bhg087
Folstein, M. F., Folstein, S. E., McHugh, P. R., 1975. “Mini-mental state”. A practical method for grading the cognitive state of patients for the clinician. J Psychiatr Res 12, 189-198. https://doi.org/10.1016/0022-3956 (75) 90026-6
Frisoni, G. B., Laakso, M. P., Beltramello, A., Geroldi, C., Bianchetti, A., Soininen, H., Trabucchi, M., 1999. Hippocampal and entorhinal cortex atrophy in frontotemporal dementia and Alzheimer's disease. Neurology 52, 91-100. https://doi.org/10.1212/WNL.52.1.91
Giesel, F. L., Hahn, H. K., Thomann, P. A., Widjaja, E., Wignall, E., von Tengg-Kobligk, H., Pantel, J., Griffiths, P. D., Peitgen, H. O., Schroder, J., Essig, M., n.d. Temporal Horn Index and Volume of Medial Temporal Lobe Atrophy Using a New Semiautomated Method for Rapid and Precise Assessment.
Gómez-Isla, T., Price, J. L., McKeel, D. W., Morris, J. C., Growdon, J. H., Hyman, B. T., 1996a. Profound loss of layer II entorhinal cortex neurons occurs in very mild Alzheimer's disease. J Neurosci 16, 4491-4500. https://doi.org/10.1523/JNEUROSCI.16-14-04491.1996
Gómez-Isla, T., Price, J. L., McKeel, D. W., Morris, J. C., Growdon, J. H., Hyman, B. T., 1996b. Profound Loss of Layer II Entorhinal Cortex Neurons Occurs in Very Mild Alzheimer's Disease. The Journal of Neuroscience 16, 4491. https://doi.org/10.1523/JNEUROSCI.16-14-04491.1996
Gosche, K. M., Mortimer, J. A., Smith, C. D., Markesbery, W. R., Snowdon, D. A., 2002. Hippocampal volume as an index of Alzheimer neuropathology. Neurology 58, 1476-1482. https://doi.org/10.1212/WNL.58.10.1476
Grochowalski, J. H., Liu, Y., Siedlecki, K. L., 2015. Aging, Neuropsychology, and Cognition A Journal on Normal and Dysfunctional Development Examining the reliability of ADAS-Cog change scores. https://doi.org/10.1080/13825585.2015.1127320
Harper, L., Bouwman, F., Burton, E. J., Barkhof, F., Scheltens, P., O'Brien, J. T., Fox, N.C., Ridgway, G. R., Schott, J. M., 2017. Patterns of atrophy in pathologically confirmed dementias: a voxelwise analysis. J Neurol Neurosurg Psychiatry 88, 908-916. https://doi.org/10.1136/jnnp-2016-314978
Harris, C. R., Millman, K. J., van der Walt, S. J., Gommers, R., Virtanen, P., Cournapeau, D., Wieser, E., Taylor, J., Berg, S., Smith, N.J., Kern, R., Picus, M., Hoyer, S., van Kerkwijk, M. H., Brett, M., Haldane, A., del Río, J. F., Wiebe, M., Peterson, P., Gérard-Marchant, P., Sheppard, K., Reddy, T., Weckesser, W., Abbasi, H., Gohlke, C., Oliphant, T. E., 2020. Array programming with NumPy. Nature 585, 357-362. https://doi.org/10.1038/S41586-020-2649-2
Hughes, C. P., Berg, L., Danziger, W. L., Coben, L. A., Martin, R. L., 1982. A new clinical scale for the staging of dementia. Br J Psychiatry 140, 566-572. https://doi.org/10.1192/BJP.140.6.566
Jack, C. R., Bennett, D. A., Blennow, K., Carrillo, M. C., Dunn, B., Haeberlein, S. B., Holtzman, D. M., Jagust, W., Jessen, F., Karlawish, J., Liu, E., Molinuevo, J. L., Montine, T., Phelps, C., Rankin, K. P., Rowe, C. C., Scheltens, P., Siemers, E., Snyder, H. M., Sperling, R., Elliott, C., Masliah, E., Ryan, L., Silverberg, N., 2018. NIA-AA Research Framework: Toward a biological definition of Alzheimer's disease. Alzheimers Dement 14, 535-562. https://doi.org/10.1016/J. JALZ.2018.02.018
Jack, C. R., Bernstein, M. A., Fox, N.C., Thompson, P., Alexander, G., Harvey, D., Borowski, B., Britson, P. J., Whitwell, J. L., Ward, C., Dale, A. M., Felmlee, J. P., Gunter, J. L., Hill, D. L. G., Killiany, R., Schuff, N., Fox-Bosetti, S., Lin, C., Studholme, C., DeCarli, C. S., Krueger, G., Ward, H. A., Metzger, G. J., Scott, K. T., Mallozzi, R., Blezek, D., Levy, J., Debbins, J. P., Fleisher, A. S., Albert, M., Green, R., Bartzokis, G., Glover, G., Mugler, J., Weiner, M. W., 2008. The Alzheimer's Disease Neuroimaging Initiative (ADNI): MRI methods. J Magn Reson Imaging 27, 685-691. https://doi.org/10.1002/JMRI.21049
Jack, C. R., Dickson, D. W., Parisi, J. E., Xu, Y. C., Cha, R. H., O'Brien, P. C., Edland, S. D., Smith, G. E., Boeve, B. F., Tangalos, E. G., Kokmen, E., Petersen, R. C., 2002. Antemortem MRI Findings Correlate with Hippocampal Neuropathology in Typical Aging and Dementia. Neurology 58, 750. https://doi.org/10.1212/WNL.58.5.750
Jack, C. R., Petersen, R. C., Xu, Y. C., Waring, S. C., O'Brien, P. C., Tangalos, E. G., Smith, G. E., Ivnik, R. J., Kokmen, E., 1997. Medial temporal atrophy on MRI in normal aging and very mild Alzheimer's disease. Neurology 49, 786-794. https://doi.org/10.1212/WNL.49.3.786
Jacobs, H. I. L., van Boxtel, M. P. J., Jolles, J., Verhey, F. R. J., Uylings, H. B. M., 2012a. Parietal cortex matters in Alzheimer's disease: An overview of structural, functional and metabolic findings. Neuroscience & Biobehavioral Reviews 36, 297-309. https://doi.org/10.1016/J.NEUBIOREV.2011.06.009
Jacobs, H. I. L., van Boxtel, M. P. J., Jolles, J., Verhey, F. R. J., Uylings, H. B. M., 2012b. Parietal cortex matters in Alzheimer's disease: an overview of structural, functional and metabolic findings. Neurosci Biobehav Rev 36, 297-309. https://doi.org/10.1016/J.NEUBIOREV.2011.06.009
Jang, J. W., Park, S. Y., Park, Y. H., Baek, M. J., Lim, J. S., Youn, Y. C., Kim, S., 2015. A comprehensive visual rating scale of brain magnetic resonance imaging: application in elderly subjects with Alzheimer's disease, mild cognitive impairment, and normal cognition. J Alzheimers Dis 44, 1023-1034. https://doi.org/10.3233/JAD-142088
Jovicich, J., Czanner, S., Greve, D., Haley, E., van der Kouwe, A., Gollub, R., Kennedy, D., Schmitt, F., Brown, G., MacFall, J., Fischl, B., Dale, A., 2006. Reliability in multi-site structural MRI studies: Effects of gradient non-linearity correction on phantom and human data. Neuroimage 30, 436-443. https://doi.org/DOI: 10.1016/j.neuroimage.2005.09.046
Khan, W., Westman, E., Jones, N., Wahlund, L. O., Mecocci, P., Vellas, B., Tsolaki, M., Kłoszewska, I., Soininen, H., Spenger, C., Lovestone, S., Muehlboeck, J. S., Simmons, A., 2015. Automated Hippocampal Subfield Measures as Predictors of Conversion from Mild Cognitive Impairment to Alzheimer's Disease in Two Independent Cohorts. Brain Topogr 28, 746-759. https://doi.org/10.1007/S10548-014-0415-1
Koikkalainen, J., Rhodius-Meester, H., Tolonen, A., Barkhof, F., Tijms, B., Lemstra, A. W., Tong, T., Guerrero, R., Schuh, A., Ledig, C., Rueckert, D., Soininen, H., Remes, A. M., Waldemar, G., Hasselbalch, S., Mecocci, P., van der Flier, W., Lötjönen, J., 2016. Differential diagnosis of neurodegenerative diseases using structural MRI data. Neuroimage Clin 11, 435-449. https://doi.org/10.1016/J.NICL.2016.02.019
Laakso, M. P., Partanen, K., Riekkinen, P., Lehtovirta, M., Helkala, E. L., Hallikainen, M., Hänninen, T., Vainio, P., Soininen, H., 1996. Hippocampal volumes in Alzheimer's disease, Parkinson's disease with and without dementia, and in vascular dementia: An MRI study. Neurology 46, 678-681. https://doi.org/10.1212/WNL.46.3.678
Laakso, M. P., Soininen, H., Partanen, K., Lehtovirta, M., Hallikainen, M., Hänninen, T., Helkala, E. L., Vainio, P., Riekkinen, P. J., 1998. MRI of the hippocampus in Alzheimer's disease: sensitivity, specificity, and analysis of the incorrectly classified subjects. Neurobiol Aging 19, 23-31. https://doi.org/10.1016/S0197-4580 (98) 00006-2
Lindeboom, J., Weinstein, H., 2004. Neuropsychology of cognitive ageing, minimal cognitive impairment, Alzheimer's disease, and vascular cognitive impairment. European Journal of Pharmacology 490, 83-86. https://doi.org/10.1016/J. EJPHAR.2004.02.046
Lu, B., Li, H.-X., Chang, Z.-K., Li, L., Chen, N.-X., Zhu, Z.-C., Zhou, H.-X., Li, X.-Y., Wang, Y.-W., Cui, S.-X., Deng, Z.-Y., Fan, Z., Yang, H., Chen, X., Thompson, P. M., Castellanos, F. X., Yan, C.-G., Initiative, for the A.D.N., 2021. A Practical Alzheimer Disease Classifier via Brain Imaging-Based Deep Learning on 85,721 Samples. bioRxiv 2020.08.18.256594. https://doi.org/10.1101/2020.08.18.256594
Ma, D., Popuri, K., Bhalla, M., Sangha, O., Lu, D., Cao, J., Jacova, C., Wang, L., Beg, M. F., 2019. Quantitative assessment of field strength, total intracranial volume, sex, and age effects on the goodness of harmonization for volumetric analysis on the ADNI database. Human Brain Mapping 40, 1507-1527. https://doi.org/10.1002/HBM.24463
Maiseli, B. J., 2021. Hausdorff Distance with Outliers and Noise Resilience Capabilities. SN Computer Science 2. https://doi.org/10.1007/S42979-021-00737-Y
Mazziotta, J. C., Toga, A. W., Evans, A., Fox, P., Lancaster, J., 1995a. A Probabilistic Atlas of the Human Brain: Theory and Rationale for Its Development: The International Consortium for Brain Mapping (ICBM). Neuroimage 2, 89-101. https://doi.org/10.1006/NIMG.1995.1012
Mazziotta, J. C., Toga, A. W., Evans, A. C., Fox, P. T., Lancaster, J. L., 1995b. Digital brain atlases. Trends in Neurosciences 18, 210-211. https://doi.org/10.1016/0166-2236 (95) 93904-C
Morris, J. C., 1993. The Clinical Dementia Rating (CDR). Neurology 43, 2412-2412-a. https://doi.org/10.1212/WNL.43.11.2412-A
Mueller, S. G., Weiner, M. W., Thal, L. J., Petersen, R. C., Jack, C., Jagust, W., Trojanowski, J. Q., Toga, A. W., Beckett, L., 2005a. The Alzheimer's disease neuroimaging initiative. Neuroimaging Clin N Am 15, 869-877. https://doi.org/10.1016/J.NIC.2005.09.008
Mueller, S. G., Weiner, M. W., Thal, L. J., Petersen, R. C., Jack, C. R., Jagust, W., Trojanowski, J. Q., Toga, A. W., Beckett, L., 2005b. Ways toward an early diagnosis in Alzheimer's disease: the Alzheimer's Disease Neuroimaging Initiative (ADNI). Alzheimers Dement 1, 55-66. https://doi.org/10.1016/J. JALZ.2005.06.003
Mukherji, D., Mukherji, M., Mukherji, N., Initiative, A. D. N., 2021. Early Detection of Alzheimer's Disease with Low-Cost Neuropsychological Tests: A Novel Predict-Diagnose Approach using Recurrent Neural Networks. medRxiv 2021.01.17.21249822. https://doi.org/10.1101/2021.01.17.21249822
Nestor, Sean M., Rupsingh, R., Borrie, M., Smith, M., Accomazzi, V., Wells, J. L., Fogarty, J., Bartha, R., 2008. Ventricular enlargement as a possible measure of Alzheimer's disease progression validated using the Alzheimer's disease neuroimaging initiative database. Brain 131, 2443-2454. https://doi.org/10.1093/BRAIN/AWN146
Nestor, Sean M, Rupsingh, R., Borrie, M., Smith, M., Accomazzi, V., Wells, J. L., Fogarty, J., Bartha, R., Initiative, the A.D.N., 2008. Ventricular enlargement as a possible measure of Alzheimer's disease progression validated using the Alzheimer's disease neuroimaging initiative database. Brain 131, 2443-2454. https://doi.org/10.1093/BRAIN/AWN146
Nie, X., Sun, Y., Wan, S., Zhao, H., Liu, R., Li, X., Wu, S., Nedelska, Z., Hort, J., Qing, Z., Xu, Y., Zhang, B., 2017. Subregional Structural Alterations in Hippocampus and Nucleus Accumbens Correlate with the Clinical Impairment in Patients with Alzheimer's Disease Clinical Spectrum: Parallel Combining Volume and Vertex-Based Approach. Front Neurol 8. https://doi.org/10.3389/FNEUR.2017.00399
O'Bryant, S. E., Waring, S. C., Cullum, C. M., Hall, J., Lacritz, L., Massman, P. J., Lupo, P. J., Reisch, J. S., Doody, R., 2008. Staging Dementia Using Clinical Dementia Rating Scale Sum of Boxes Scores: A Texas Alzheimer's Research Consortium Study. Arch Neurol 65, 1091. https://doi.org/10.1001/ARCHNEUR.65.8.1091
Ott, B. R., Cohen, R. A., Gongvatana, A., Okonkwo, O. C., Johanson, C. E., Stopa, E. G., Donahue, J. E., Silverberg, G. D., 2010. Brain ventricular volume and cerebrospinal fluid biomarkers of Alzheimer's disease. J Alzheimers Dis 20, 647. https://doi.org/10.3233/JAD-2010-1406
Petersen, R. C., Aisen, P. S., Beckett, L. A., Donohue, M. C., Gamst, A. C., Harvey, D. J., Jack, C. R., Jagust, W. J., Shaw, L. M., Toga, A. W., Trojanowski, J. Q., Weiner, M. W., 2010. Alzheimer's Disease Neuroimaging Initiative (ADNI): Clinical characterization. Neurology 74, 201. https://doi.org/10.1212/WNL.0B013E3181CB3E25
Pinto, M. F., Leal, A., Lopes, F., Pais, J., Dourado, A., Sales, F., Martins, P., Teixeira, C. A., 2022. On the clinical acceptance of black-box systems for EEG seizure prediction. Epilepsia Open 7. https://doi.org/10.1002/EPI4.12597
Popuri, K., Ma, D., Wang, L., Beg, M. F., 2020a. Using machine learning to quantify structural MRI neurodegeneration patterns of Alzheimer's disease into dementia score: Independent validation on 8,834 images from ADNI, AIBL, OASIS, and MIRIAD databases. Hum Brain Mapp 41, 4127-4147. https://doi.org/10.1002/HBM.25115
Popuri, K., Ma, D., Wang, L., Beg, M. F., 2020b. Using machine learning to quantify structural MRI neurodegeneration patterns of Alzheimer's disease into dementia score: Independent validation on 8,834 images from ADNI, AIBL, OASIS, and MIRIAD databases. Hum Brain Mapp 41, 4127-4147. https://doi.org/10.1002/HBM.25115
Rabinovici, G. D., Seeley, W. W., Kim, E. J., Gorno-Tempini, M. L., Rascovsky, K., Pagliaro, T. A., Allison, S. C., Halabi, C., Kramer, J. H., Johnson, J. K., Weiner, M. W., Forman, M. S., Trojanowski, J. Q., Dearmond, S. J., Miller, B. L., Rosen, H. J., 2007. Distinct MRI Atrophy Patterns in Autopsy-Proven Alzheimer's Disease and Frontotemporal Lobar Degeneration. Am J Alzheimers Dis Other Demen 22, 474. https://doi.org/10.1177/1533317507308779
Rallabandi, V. P. Subramanyam, Tulpule, K., Gattu, M., 2020. Automatic classification of cognitively normal, mild cognitive impairment and Alzheimer's disease using structural MRI analysis. Informatics in Medicine Unlocked 18, 100305. https://doi.org/10.1016/J. IMU.2020.100305
Rallabandi, V P. Subramanyam, Tulpule, K., Gattu, M., 2020. Automatic classification of cognitively normal, mild cognitive impairment and Alzheimer's disease using structural MRI analysis. Informatics in Medicine Unlocked 18, 100305. https://doi.org/10.1016/J. IMU.2020.100305
Reuter, M., Rosas, H. D., Fischl, B., 2010. Highly Accurate Inverse Consistent Registration: A Robust Approach. Neuroimage 53, 1181-1196. https://doi.org/10.1016/j.neuroimage.2010.07.020
Rosen, W. G., Mohs, R. C., Davis, K. L., 1984. A new rating scale for Alzheimer's disease. Am J Psychiatry 141. https://doi.org/10.1176/AJP.141.11.1356
Salvatore, C., Cerasa, A., Castiglioni, I., 2018. MRI characterizes the progressive course of AD and predicts conversion to Alzheimer's dementia 24 months before probable diagnosis. Frontiers in Aging Neuroscience 10, 135. https://doi.org/10.3389/FNAGI.2018.00135/BIBTEX
Schott, J. M., Price, S. L., Frost, C., Whitwell, J. L., Rossor, M. N., Fox, N.C., 2005. Measuring atrophy in Alzheimer disease. Neurology 65, 119-124. https://doi.org/10.1212/01. WNL.0000167542.89697.0F
Segonne, F., Dale, A. M., Busa, E., Glessner, M., Salat, D., Hahn, H. K., Fischl, B., 2004. A hybrid approach to the skull stripping problem in MRI. Neuroimage 22, 1060-1075. https://doi.org/DOI: 10.1016/j.neuroimage.2004.03.032
Segonne, F., Pacheco, J., Fischl, B., 2007. Geometrically accurate topology-correction of cortical surfaces using nonseparating loops. IEEE Trans Med Imaging 26, 518-529.
Silbert, L. C., Quinn, J. F., Moore, M. M., Corbridge, E., Ball, M. J., Murdoch, G., Sexton, G., Kaye, J. A., 2003. Changes in premorbid brain volume predict Alzheimer's disease pathology. Neurology 61, 487-492. https://doi.org/10.1212/01. WNL.0000079053.77227.14
Sørensen, L., Igel, C., Liv Hansen, N., Osler, M., Lauritzen, M., Rostrup, E., Nielsen, M., 2016. Early detection of Alzheimer's disease using MRI hippocampal texture. Human Brain Mapping 37, 1148-1161. https://doi.org/10.1002/HBM.23091
Stein, J. L., Medland, S. E., Vasquez, A. A., Hibar, D. P., Senstad, R. E., Winkler, A. M., Toro, R., Appel, K., Bartecek, R., Bergmann, Ø., Bernard, M., Brown, A. A., Cannon, D. M., Chakravarty, M. M., Christoforou, A., Domin, M., Grimm, O., Hollinshead, M., Holmes, A. J., Homuth, G., Hottenga, J.-J., Langan, C., Lopez, L. M., Hansell, N. K., Hwang, K. S., Kim, S., Laje, G., Lee, P. H., Liu, X., Loth, E., Lourdusamy, A., Mattingsdal, M., Mohnke, S., Maniega, S. M., Nho, K., Nugent, A. C., O'Brien, C., Papmeyer, M., Pütz, B., Ramasamy, A., Rasmussen, J., Rijpkema, M., Risacher, S. L., Roddey, J. C., Rose, E. J., Ryten, M., Shen, L., Sprooten, E., Strengman, E., Teumer, A., Trabzuni, D., Turner, J., van Eijk, K., van Erp, T. G. M., van Tol, M.-J., Wittfeld, K., Wolf, C., Woudstra, S., Aleman, A., Alhusaini, S., Almasy, L., Binder, E. B., Brohawn, D. G., Cantor, R. M., Carless, M. A., Corvin, A., Czisch, M., Curran, J. E., Davies, G., de Almeida, M. A. A., Delanty, N., Depondt, C., Duggirala, R., Dyer, T. D., Erk, S., Fagerness, J., Fox, P. T., Freimer, N. B., Gill, M., Göring, H. H. H., Hagler, D. J., Hoehn, D., Holsboer, F., Hoogman, M., Hosten, N., Jahanshad, N., Johnson, M. P., Kasperaviciute, D., Kent Jr, J. W., Kochunov, P., Lancaster, J. L., Lawrie, S. M., Liewald, D. C., Mandl, R., Matarin, M., Mattheisen, M., Meisenzahl, E., Melle, I., Moses, E. K., Mühleisen, T. W., Nauck, M., Nothen, M. M., Olvera, R. L., Pandolfo, M., Pike, G. B., Puls, R., Reinvang, I., Rentería, M. E., Rietschel, M., Roffman, J. L., Royle, N. A., Rujescu, D., Savitz, J., Schnack, H. G., Schnell, K., Seiferth, N., Smith, C., Steen, V. M., Valdés Hernández, M. C., van den Heuvel, M., van der Wee, N.J., van Haren, N. E. M., Veltman, J. A., Völzke, H., Walker, R., Westlye, L. T., Whelan, C. D., Agartz, I., Boomsma, D. I., Cavalleri, G. L., Dale, A. M., Djurovic, S., Drevets, W. C., Hagoort, P., Hall, J., Heinz, A., Jack Jr, C. R., Foroud, T. M., le Hellard, S., Macciardi, F., Montgomery, G. W., Poline, J. B., Porteous, D. J., Sisodiya, S. M., Starr, J. M., Sussmann, J., Toga, A. W., Veltman, D. J., Walter, H., Weiner, M. W., Initiative, A. D.N., Consortium, E., Consortium, I., Group, S. Y. S., Bis, J. C., Ikram, M. A., Smith, A. v, Gudnason, V., Tzourio, C., Vernooij, M. W., Launer, L. J., DeCarli, C., Seshadri, S., Consortium, C. for H. and A. R. in G. E., Andreassen, O. A., Apostolova, L. G., Bastin, M. E., Blangero, J., Brunner, H. G., Buckner, R. L., Cichon, S., Coppola, G., de Zubicaray, G. I., Deary, I. J., Donohoe, G., de Geus, E. J. C., Espeseth, T., Fernández, G., Glahn, D. C., Grabe, H. J., Hardy, J., Hulshoff Pol, H. E., Jenkinson, M., Kahn, R. S., McDonald, C., McIntosh, A. M., McMahon, F. J., McMahon, K. L., Meyer-Lindenberg, A., Morris, D. W., Müller-Myhsok, B., Nichols, T. E., Ophoff, R. A., Paus, T., Pausova, Z., Penninx, B. W., Potkin, S. G., Sämann, P. G., Saykin, A. J., Schumann, G., Smoller, J. W., Wardlaw, J. M., Weale, M. E., Martin, N. G., Franke, B., Wright, M. J., Thompson, P. M., Consortium, E. N. I. G. through M.-A., 2012. Identification of common variants associated with human hippocampal and intracranial volumes. Nat Genet 44, 552-561. https://doi.org/10.1038/ng.2250
Thompson, P. M., Hayashi, K. M., de Zubicaray, G., Janke, A. L., Rose, S. E., Semple, J., Herman, D., Hong, M. S., Dittmer, S. S., Doddrell, D. M., Toga, A. W., 2003. Dynamics of Gray Matter Loss in Alzheimer's Disease. The Journal of Neuroscience 23, 994. https://doi.org/10.1523/JNEUROSCI.23-03-00994.2003
Thompson, P. M., Hayashi, K. M., Dutton, R. A., Chiang, M. C., Leow, A. D., Sowell, E. R., de Zubicaray, G., Becker, J. T., Lopez, O. L., Aizenstein, H. J., Toga, A. W., 2007. Tracking Alzheimer's Disease. Ann N Y Acad Sci 1097, 183. https://doi.org/10.1196/ANNALS.1379.017
Vemuri, P., Gunter, J. L., Senjem, M. L., Whitwell, J. L., Kantarci, K., Knopman, D. S., Boeve, B. F., Petersen, R. C., Jack, C. R., n.d. Alzheimer's Disease Diagnosis in Individual Subjects using Structural MR Images: Validation Studies.
Vemuri, P., Wiste, H. J., Weigand, S. D., Shaw, L. M., Trojanowski, J. Q., Weiner, M. W., Knopman, D. S., Petersen, R. C., Jack, C. R., 2009a. MRI and CSF biomarkers in normal, MCI, and AD subjects: predicting future clinical change. Neurology 73, 294-301. https://doi.org/10.1212/WNL.0B013E3181AF79FB
Vemuri, P., Wiste, H. J., Weigand, S. D., Shaw, L. M., Trojanowski, J. Q., Weiner, M. W., Knopman, D. S., Petersen, R. C., Jack, C. R., 2009b. MRI and CSF biomarkers in normal, MCI, and AD subjects: Diagnostic discrimination and cognitive correlations. Neurology 73, 287-293. https://doi.org/10.1212/WNL.0B013E3181AF79E5
Vercelletto, M., Martinez, F., Lanier, S., Magne, C., Jaulin, P., Bourin, M., 2002. How to define treatment success using cholinesterase inhibitors. Int J Geriatr Psychiatry 17, 388-390. https://doi.org/10.1002/GPS.612
WHO, 2021. Dementia Fact Sheet [WWW Document]. URL https://www.who.int/news-room/fact-sheets/detail/dementia (accessed 2.15.22).
Yin, C., Li, S., Zhao, W., Feng, J., 2013. Brain imaging of mild cognitive impairment and Alzheimer's disease. Neural Regen Res 8, 435-444. https://doi.org/10.3969/j.issn.1673-5374.2013.05.007
Zanchi, D., Giannakopoulos, P., Borgwardt, S., Rodriguez, C., Haller, S., 2017. Hippocampal and Amygdala Gray Matter Loss in Elderly Controls with Subtle Cognitive Decline. Frontiers in Aging Neuroscience 9.doi: 10.1002/hbm.24463.

### Implementations

The following implementations are exemplary only and do not limit the scope of the present disclosure or the appended claims.

Implementation 1. A method comprising: receiving, from at least one computing device, a set of magnetic resonance (MR) images related to a brain region; determining, from the MR images, a volume of the brain region associated with cognitive decline; determining a benchmark based on a biomarker metric for a neurodegenerative disease, wherein the biomarker metric is based on a cohort of data; and generating an assessment score, based on the benchmark, to predict a cognitive state.

Implementation 2. The method of Implementation 1, wherein the brain region is at least one of a cortical region and a sub-cortical region.

Implementation 3. The method of any of Implementations 1-2, wherein the biomarker metric is adjusted hippocampal volume (AHV).

Implementation 4. The method of any of Implementations 1-3, wherein the cognitive state is at least one of cognitively normal (CN), mild cognitive impairment (MCI), or Alzheimer's Disease (AD).

Implementation 5. The method of any of Implementations 1-4, further comprising determining a severity level, based on a second biomarker, when the cognitive state is indicative of Alzheimer's Disease (AD).

Implementation 6. The method of any of Implementations 1-5, wherein the cohort of data comprises a set of demographic data.

Implementation 7. The method of Implementation 6, wherein the demographics include at least one of a class, an age, a gender, and an education level.

Implementation 8. The method of any one of Implementations 1-7, wherein determining the benchmark comprises applying a machine learning algorithm.

Implementation 9. The method of any of Implementations 1-8, wherein determining the benchmark further comprises applying a distance metric to the biomarker metric and cohort of data.

Implementation 10. The method of Implementation 9, wherein the distance metric is a Hausdorff metric.

Implementation 11. A computing system, comprising a processor and a memory storing instructions that, when executed by the processor, causes the computing system to execute the method of any of Implementations 1-10.

Those skilled in the art also will readily appreciate that many additional modifications are possible in the exemplary implementation without materially departing from the novel teachings and advantages of the invention. Accordingly, any such modifications are intended to be included within the scope of this invention as defined by the following exemplary claims.

What is claimed:

1. A method, comprising:
receiving, from at least one computing device, a set of magnetic resonance (MR) images related to a brain region;
determining, from the MR images, a volume of the brain region associated with cognitive decline;
determining a benchmark based on a biomarker metric for a neurodegenerative disease, wherein the biomarker metric compares the volume of the brain region to a cohort of data by treating the volume as a vector in n-dimensional space;
generating an assessment score, based on the benchmark, to predict a cognitive state.

2. The method of claim 1, wherein the biomarker metric is computed based on a Z-weighted Euclidean distance (ZWE), according to:
$$ZWE = {{\sum }_{i = 1}^{NR} [ ( {w}_{i}^{T} - {w}_{i}^{S} ) {z}_{i} ]}^{2} .$$

3. The method of claim 2, wherein z; refers to an average of z-transformed p-value across possible pairwise comparisons for a given region, i, and NR denotes the total number of regions, and wherein the possible pairwise comparisons include cognitively normal versus mild cognitive impairment, cognitively normal versus Alzheimer's Disease, and mild cognitive impairment versus Alzheimer's Disease.

4. The method of claim 1, wherein the brain region is at least one of a cortical region and a sub-cortical region.

5. The method of claim 1, wherein the biomarker metric is adjusted hippocampal volume (AHV).

6. The method of claim 1, wherein the cognitive state is at least one of cognitively normal (CN), mild cognitive impairment (MCI), or Alzheimer's Disease (AD).

7. The method of claim 1, further comprising determining a severity level, based on a second biomarker, when the cognitive state is indicative of Alzheimer's Disease (AD).

8. The method of claim 1, wherein the cohort of data comprises a set of demographic data.

9. The method of claim 8, wherein the demographics include at least one of a class, an age, a gender, and an education level.

10. The method of claim 1, wherein determining the benchmark comprises applying a machine learning algorithm trained on associations between biomarker metrics and neurodegenerative disease states.

11. The method of claim 1, wherein determining the benchmark further comprises applying a distance metric to the biomarker metric and cohort of data.

12. The method of claim 11, wherein the distance metric comprises at least one of a Euclidean metric, a Hausdorff metric, and a Frechet metric.

13. A computing system, comprising a processor and a memory storing instructions that, when executed by the processor, causes the computing system to execute the method of claim 1.

14. A non-transitory computer readable medium comprising instructions, which when executed by a processor cause a computing device to at least:
receive, from at least one computing device, a set of magnetic resonance (MR) images related to a brain region;
determine, from the MR images, a volume of the brain region associated with cognitive decline;
determine a benchmark based on a biomarker metric for a neurodegenerative disease, wherein the biomarker metric compares the volume of the brain region to a cohort of data by treating the volume as a vector in n-dimensional space; and
generate an assessment score, based on the benchmark, to predict a cognitive state.

15. The non-transitory computer-readable medium of claim 14, wherein the biomarker metric is computed based on a Z-weighted Euclidean distance (ZWE), according to:
$$ZWE = {{\sum }_{i = 1}^{NR} [ ( {w}_{i}^{T} - {w}_{i}^{S} ) {z}_{i} ]}^{2} ,$$
where zᵢ refers to an average of z-transformed p-value across possible pairwise comparisons for a given region, i, and NR denotes the total number of regions.

16. The non-transitory computer-readable medium of claim 15, wherein the possible pairwise comparisons include cognitively normal versus mild cognitive impairment, cognitively normal versus Alzheimer's Disease, and mild cognitive impairment versus Alzheimer's Disease.

17. The non-transitory computer-readable medium of claim 14, further comprising instructions, which when executed by a process, cause a computing device to:
apply a machine learning algorithm trained on associations between biomarker metrics and neurodegenerative disease states.

18. The non-transitory computer-readable medium of claim 14, further comprising instructions, which when executed by a process, cause a computing device to
determine the benchmark further comprises applying a distance metric to the biomarker metric and cohort of data, wherein the distance metric comprises at least one of a Euclidean metric, a Hausdorff metric, and a Frechet metric.

19. The non-transitory computer-readable medium of claim 14, wherein the cohort of data comprises a set of demographic data, and wherein the demographics include at least one of a class, an age, a gender, and an education level.

20. The non-transitory computer-readable medium of claim 14, wherein the brain region is at least one of a cortical region and a sub-cortical region.
